(19) United States
(12) Patent Application Publication
(10) Pub. No.: US 2018/0271888 A1
(43) Pub. Date: Sep. 27, 2018

(54) COMPOSITIONS AND METHODS FOR PARKINSON'S DISEASE THERAPY

(71) Applicant: Daryl Davies, Los Angeles, CA (US)

(72) Inventor: Daryl Davies, Los Angeles, CA (US)

(21) Appl. No.: 15/933,215

(22) Filed: Mar. 22, 2018

Related U.S. Application Data
(60) Provisional application No. 62/475,798, filed on Mar. 23, 2017.

(51) Int. Cl. A61K 31/7048; A61K 31/365; A61K 31/197; A61P 25/28

(52) U.S. Cl. CPC A61K 31/7048; A61P 25/28; A61K 31/197; A61K 31/365

(57) ABSTRACT

This disclosure provides methods for the treatment of one or more of: Parkinson's disease (PD); PD symptoms; movement disorders; neurodegenerative diseases linked to changes in dopamine, dopamine signaling, or dopamine expression, in a subject in need thereof comprising administering to the subject an effective amount of an agent that potentiates dopaminergic neurotransmission. Also provided is a method for selecting a candidate agent for the treatment of Parkinson's disease (PD); PD symptoms; and neurodegenerative diseases linked to changes in dopamine, dopamine signaling, or dopamine expression.

### CROSS REFERENCE TO RELATED APPLICATIONS

This application claims the benefit of under 35 U.S.C. § 119(e) to U.S. Provisional Application Ser. No. 62/475,798, filed Mar. 23, 2017, the content of which is incorporated by reference herein in its entirety.

### STATEMENT OF GOVERNMENT SUPPORT

This invention was made with government support under the Grant No. 8UL1TR000130, awarded by the National Institute for Health/National Center for Advancing Translational Sciences (NIH/NCATS) and Grant No. R01 AA022448, awarded by the National Institute for Health/National Institute on Alcohol Abuse and Alcoholism (NIH/NIAAA). Accordingly, the U.S. Government has certain rights to the invention.

### BACKGROUND

Parkinson's disease (PD) is a neurodegenerative disorder that involves motor deficits. Currently there is no demonstrated neuroprotective or disease-modifying therapy. Patients diagnosed with PD undergo L-DOPA treatment to alleviate some of the neurodegenerative effects including tremors, rigidity and bradykinesia. Unfortunately, the benefit of L-DOPA wears off over time. New therapies are needed. This disclosure satisfies this need and provides related advantages as well.

### SUMMARY

Provided herein are compositions, methods and formulations for the treatment of one or more of: Parkinson's disease (PD); PD symptoms; movement disorders; and neurodegenerative diseases linked to changes in dopamine, dopamine signaling, or dopamine expression, in a subject in need thereof. The methods comprise, or alternatively consist essentially of, or yet further consist of, administering to the subject an effective amount of an agent that potentiates dopaminergic neurotransmission. In one aspect, this disclosure provides methods for the treatment of PD, PD symptoms, and related disorders (e.g., neurodegenerative diseases linked to changes in dopamine), the methods comprising, or alternatively consisting essentially of, or yet further consisting of, the administration of an effective amount of ivermectin (IVM), an IVM-analogue, a P2X4R allosteric modulator or a related avermectin compound, e.g., moxidectin (MOX), that in one aspect are further combined with known dopamine enhancing or PD therapies, e.g., L-DOPA (i.e., as an adjunct therapy). In another aspect the compounds are administered independent of L-DOPA as a therapy to help alleviate some of the neurodegenerative effects of PD and/or other neurodegenerative diseases linked to changes in dopamine. Also provided are compositions comprising an effective amount of the agent that potentiates dopaminergic neurotransmission, alone or in combination with L-DOPA or other adjuvant therapies. The compositions can be formulated for ease of administration for the treatment of the disease or disorder.

Also provided herein is a method for selecting a candidate agent for the treatment of one or more of: Parkinson's disease (PD); PD symptoms; and neurodegenerative diseases linked to changes in dopamine, dopamine signaling, or dopamine expression, in a subject in need thereof, the method comprising contacting a compound with a cell expressing a P2X4 receptor under conditions for binding of the candidate agent to the P2X4 receptor and selecting the compound as the candidate agent if it potentiates P2X4 receptor activity.

The compositions and methods of this disclosure are based on Applicant's discovery that administration of ivermectin (IVM) significantly increased L-DOPA induced rotations using a 6-hydroxydopamine (6-OHDA) lesion model in rodents. Notably, the 6-OHDA lesion model is routinely used for screening of compounds for various CNS diseases including PD. Applicant also discovered that the number of L-DOPA induced contralateral rotations were significantly less in male P2X4 receptor (P2X4R) knock out mice compared to wildtype (WT) control mice. Thus, without being bound by theory and to the best of Applicant's knowledge, P2X4Rs function can modulate dopaminergic neurotransmission. In support of this, western blot analysis of brain samples from WT and P2X4R KO male mice were undertaken and showed altered expressions of several important dopaminergic proteins including: D1 dopamine receptors (DA1), D2 dopamine receptors (DA2), dopamine transporter (DAT), and tyrosine hydroxylase (TH) in critical brain regions of the dopamine circuitry. Moreover, administration of ivermectin (IVM), which potentiates P2X4Rs and GABAARs, concurrently with L-DOPA to male WT mice, significantly increased in number of rotations as opposed to L-DOPA alone. When testing the effect of these combined agents on P2X4R KO male mice, a reduction in the enhancement of rotations as compared to concurrent administration of these agents to WT male mice was observed. Thus, it is shown that P2X4Rs can play a role in modulating dopamine neurotransmission and activity, and that IVM and other related avermectin compounds can be used for treatment of diseases linked to changes in dopamine signalling and/or dopamine expression.

### BRIEF DESCRIPTION OF THE FIGURES

FIGS. 1A-1B: P2X4R KO mice exhibited significant increases in TH protein density in the dorsal striatum, but no changes in the ventral striatum [FIG. 1A and FIG. 1B (i)]; increased dopamine transporter (DAT) protein density in both parts of the striatum [FIG. 1A and FIG. 1B (ii)]; increased D1R [FIG. 1A and FIG. 1B (iii)] and D2R [FIG. 1A and FIG. 1B (iv)] protein densities in the ventral, but no change in the dorsal striatum. The protein levels of dopamine markers were normalized to β-actin and expressed as arbitrary units (AU). The average of densitometry value of wildtype (WT) samples was arbitrarily normalized to 1. P2X4R KO samples were normalized by dividing each value by the average of WT samples and presented as fold change of P2X4R KO versus WT in that membrane. Values represent mean±SEM 5-8 WT, 7-8 P2X4R KO mice for TH, DAT analyses, and 11-12 WT and 12-13 P2X4R KO for D1Rs and D2Rs analyses. Two representative bands from each genotype from the same membrane are shown. *p<0.05, **p<0.01 versus WT controls.

FIGS. 2A-2B: P2X4R KO mice exhibited increased dopamine and cyclic AM pregulated phosphoprotein of 32 kDa (DARPP-32) phosphorylation in the dorsal striatum, but a decrease in the ventral striatum [FIG. 2A and FIG. 2B (i)]; no changes in extracellular regulated kinase 1/2 (ERK 1/2) phosphorylation in dorsal or ventral striatum of P2X4R KO mice [a and b (ii)]; increased phosphorylation of cyclic-AMP-response element binding protein (CREB) in the ventral, but not in the dorsal striatum [FIG. 2A and FIG. 2B(iii)]. Details of normalization and analyses are presented in FIG. 1. Values represent mean±SEM 3-6 wildtype (WT) and 4-8 P2X4R KO mice for DARPP-32 analysis; 6-8 WT and P2X4R KO for ERK1/2 and CREB analyses. Two representative bands from each genotype from the same membrane are shown. *p<0.05, ***p<0.001 versus WT counterparts. Unpaired Student's t-test.

FIGS. 3A-3B: Ivermectin (IVM) (5 mg/kg) significantly up-regulated dopamine and cyclic AMP regulated phosphoprotein of 32 kDa (DARPP-32) phosphorylation [FIG. 3A and FIG. 3B (i)] via P2X4R potentiation in the dorsal striatum. IVM increased extracellular regulated kinasel/2 (ERK 1/2) phosphorylation independent of P2X4R function [FIG. 3A and FIG. 3B (ii)]. No effect of IVM on cyclic-AMP-response element binding protein (CREB) phosphorylation [FIG. 3A and FIG. 3B (iii)]. The average of densitometry value of vehicle-treated wildtype (WT) samples was arbitrarily normalized to 1. The IVM-treated WT mice, vehicle- and IVM-treated P2X4R KO were normalized by dividing each value by the average of the vehicle-treated WT samples. The data are presented as fold change of IVM-treated WT, P2X4R KO- and vehicle-treated P2X4R KO samples versus vehicle treated WT samples in that membrane. Values represent mean±SEM from 4 to 6 WT and P2X4R KO mice per treatment group. Two representative bands from each treatment group are shown. *p<0.05 versus vehicle-treated WT group, Bonferroni post hoc test. **p<0.01 versus vehicle treated WT mice.

FIGS. 4A-4B: Ivermectin (IVM) (5 mg/kg) significantly affected dopamine and cyclic AMP regulated phosphoprotein of 32 kDa (DARPP-32) [FIG. 4A and FIG. 4B (i)] but not, extracellular regulated kinasel/2 (ERK 1/2) [FIG. 4A and FIG. 4B (ii)] or cyclic-AMP-response element binding protein (CREB) phosphorylation [FIG. 4A and FIG. 4B (iii)], via P2X4R potentiation in the ventral striatum. Details of normalization and analyses are presented in FIG. 3. Values represent mean±SEM from 4 to 6 wildtype (WT) and P2X4R KO mice per treatment group. Two representative bands from each treatment group are shown. *p<0.05 versus vehicle-treated WT group, Bonferroni post hoc test. **p<0.01 versus vehicle treated WT mice.

FIGS. 5A-5B: SCH-23390 (1 mg/kg) and raclopride (3 mg/kg) significantly increased prepulse inhibition of acoustic startle reflex in P2X4R KO mice (FIG. 5A) without any changes in startle amplitude (FIG. 5B). There were no changes in prepulse inhibition (PPI) in wildtype (WT) mice upon treatment with both antagonists (FIG. 5A). Values represent the mean of APPI and mean startle amplitude±SEM from 14 WT (saline), 15 WT (SCH 23390 and raclopride), 14 P2X4R KO (saline), and 16 P2X4R KO (SCH-23390 and raclopride). *p<0.05 versus saline-treated WT mice, ##p<0.01, ###p<0.001 versus saline-treated P2X4R KO mice, Bonferroni post hoc test.

FIGS. 6A-6B: L-DOPA (5 mg/kg)-induced rotational behavior is significantly attenuated in P2X4R KO mice. IVM (5 mg/kg) significantly potentiated L-DOPA's effect on the number of contralateral turns in wildtype (WT) and P2X4R KO mice (FIG. 6A). IVM's ability to enhance L-DOPA-induced motor behavior was significantly altered in P2X4R KO mice (FIG. 6B). Values on the y-axis represent the mean of number of contralateral turns per 10-minute interval±SEM from 14 WT, 8 P2X4R KO. *p<0.05, ***p<0.001 versus L-DOPA-treated WT mice, ##p<0.01 versus L-DOPA-treated P2X4R KO mice for (a), *p<0.05 versus WT mice for (b), Bonferroni post hoc test.

FIGS. 7A-7B: No changes in DA levels in the dorsal (FIG. 7A) and ventral striatum (FIG. 7B) of P2X4R KO mice as compared to WT controls. Values represent mean±SEM for 13 WT, 12 P2X4R KO mice in dorsal striatum and 12 WT, 11 P2X4R KO mice in ventral striatum.

FIGS. 8A-8B: Stereotaxic injection of 6-OHDA (4 mg/ml) induced destruction of DA neurons in the ventral mesencephelon (FIG. 8A) and TH density in the striatum (FIG. 8B) of both WT and P2X4R KO to similar extent. U=unlesioned, L=lesioned. Values represent mean±SEM for 4 mice per genotype. **P<0.01, *** P<0.001 versus unlesioned side of striatum, Student t-test.

FIG. 9: IVM (5 mg/kg) induced ipsilateral rotations in 6-OHDA WT mice that were statistically significant from sham WT controls. Values on the y-axis represent mean of number of ipsilateral rotations for a period of 2 hrs post IVM treatment±SEM for 7 6-OHDA WT and 4 sham controls. ** P<0.01,*** P<0.001 versus sham WT controls, Bonferroni post hoc test.

FIGS. 10A-10D: P2X4R KO mice exhibited significantly higher 10E intake compared to WT controls (FIG. 10A) and tended to have higher total fluid intake (FIG. 10D) without any significant changes in 10E preference (FIG. 10B) or water intake (FIG. 10C). For each week, the 10E intake, preference, and water intake was measured as an average of 5 days. Values represent mean±SEM for a duration of 5 days each week for eight WT and 10 P2X4R KO mice. *p<0.05 versus WT mice, two-way Analysis of variance (ANOVA).

FIGS. 11A-11C: Microglial BV-2 cells transinfected with LV-shRNA-p2rx4 reduced P2X4R expression by 68% and 62% as compared to non-treated cells (NT) and LV-alone treated cells, respectively. (FIG. 11A). Stereotaxic injection of LV-shRNA-p2rx4 in NAc core significantly reduced P2X4R expression as compared to naïve mice and LV-alone infused mice, respectively, after 14 (FIG. 11B) and 42 days (FIG. 11C). Values represent mean±SEM for 3-8 mice per treatment group for (FIG. 11B) and (FIG. 11C). **p<0.01 versus non-treated cells, #p<0.05 versus LV-alone treated cells for (A). *p<0.05, **p<0.01 versus naïve controls, #p<0.05, ##p<0.01 versus LV-alone group for (FIG. 11B) and (FIG. 11C), Tukey's post hoc test.

FIGS. 12A-12D: The LV-shRNA-p2rx4 group exhibited significantly higher 10E intake as compared to mice infused with lentvirus (LV) alone. (FIG. 12A). No significant changes in 10E preference (FIG. 12B), water intake (FIG. 12C), or total fluid intake (FIG. 12D) between the groups. Values represent mean±SEM for a duration of 5 days for each week for 10 mice infused with LV alone and 14 mice infused with LV-shRNA-p2rx4. *p<0.05, versus LV-alone infused mice, two-way ANOVA.

FIG. 13: The contralateral rotations per ten minutes over time in 5 mg/kg L-DOPA treated mice (upper most line at 0 min), 5 mg/kg L-DOPA and IVM 5 mg/kg treated mice (middle line at 0 min), and 5 mg/kg L-DOPA and MOX 2.5 mg/kg treated mice (bottom line at 0 min).

FIG. 14A-14B: In FIG. 14A the 3,4-Dihydroxyphenylacetic acid (DOPAC)level changes in the presence of various doses of MOX in mice is shown; left most bar is 0 mg/kg, 2^{nd} bar from left is 1 mg/kg, 3^{rd} bar from left is 2.5 mg/kg and right most bar is 5 mg/kg. In (FIG. 14B) is shown the DA (dopamine) level changes in the presence of various doses of MOX in mice; the left most bar is 0 mg/kg, 2^{nd} bar from left is 1 mg/kg, 3^{rd} bar from left is 2.5 mg/kg and right most bar is 5 mg/kg.

### DETAILED DESCRIPTION

Throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

As used herein, the term “reporter” means an element on or within an isolated cell having a characteristic (e.g., activity, expression, localization, interaction, modification, etc.) which is one or more of: dependent upon, correlates with, or activated by physiological changes or conditions of the cell.

“Parkinson's disease” or “PD” intends the progressive disorder of the nervous system that affects movement. It develops gradually, sometimes starting with a barely noticeable tremor in just one hand. But while a tremor may be the most well-known sign of Parkinson's disease, the disorder also commonly causes stiffness or slowing of movement.

PD symptom includes, clinical and sub-clinical symptoms of PD, including without limitation tremor, stiffness, slowness, impaired balance, and a shuffling gait.

“Parkinsonian syndrome,” “Parkinson's disease (PD) symptoms” and the like, refers to symptoms of Parkinson's disease (such as slow movements and tremors). Other symptoms include stiff muscles and difficulty with balance and walking.

As used herein, the term “movement disorder” intends a neurological disturbance that involves one or more muscles or muscle groups. Non-limiting examples include Parkinson's disease, Huntington's Chorea, progressive supranuclear palsy, Wilson's disease, Tourette's syndrome, epilepsy, and various chronic tremors, tics and dystonias.

As used herein, the term “neurodegenerative disease linked to changes in dopamine (DA), dopamine signaling or dopamine expression” intends abnormalities in the dopaminergic system and its receptors in the basal ganglia structures that are the basis Parkinson's disease (PD), however DA also participates in other neurodegenerative disorders such as Huntington disease (HD), Alzheimer's, attention deficit disorder, cognitive impairment, confusion, depersonalization, depression, inattentiveness, low libido, memory impairment, sleepiness, and multiple sclerosis (MS). These conditions are known in the art, and described for example in Rangel-Barajas et al. (2015) “Dopamine Receptors and Neurodegeneration,” Aging Dis. 6(5):349-368 and mentalhealthdaily.com/2015/04/02/low-dopamine-levels-symptoms-adverse-reactions, last accessed on Mar. 23, 2017.

“Bradykinesia” is slow movement often associated with impaired ability to adjust the body's position.

“Encephalitis” is inflammation of the brain.

“Concussion” is a head injury with a temporary loss in brain function.

“Dystonia” is movement disorder in which a person's muscles contract uncontrollably. Symptoms of the disorder include a dragging leg, a cramping foot, an involuntary pulling of the neck, uncontrollable blinking or difficulties with speaking. “Dopamine-responsive dystonia” is a rare form of genetic dystonia.

“Analogue” may refer to a structural analogue or functional analogue of a chemical compound. Structural analogues share similarity in chemical structure. Functional analogues share similarity in their physical, chemical, biochemical or pharmacological properties.

Potentiation of dopaminergic neurotransmission referred to herein refers to inducing an increase in dopaminergic neurotransmission. Increases in dopaminergic neurotransmission may be identified through any of the methods commonly known in the art, including, but not limited to positron tomography scanning using radioisotopes, measurement of spontaneous eye-blink rate in a subject, rate of contralateral rotations, microdialysis and/or detection of catecholamines in bodily fluids using an analytical method, for example, gas chromatography mass spectrometry (GCMS).

An “agent” intends a small molecule or molecules, or large molecule or molecules or biologic or biologics or any combination thereof, e.g., inhibitory RNA or antibody, antibody fragment or modification thereof. Non-limiting examples include ivermectin, an ivermectin analogue, moxidectin, a moxidectin analogue and P2X4R allosteric modulators, e.g., zinc.

“Candidate agent” refers to a compound for the treatment of one or more of: Parkinson's disease (PD); PD symptoms; or neurodegenerative diseases linked to changes in dopamine, dopamine signaling, or dopamine expression, in a subject in need thereof.

“Allosteric modulator” is a substance which indirectly influences the effects of a receptor agonist or inverse agonist at its receptor protein target. Allosteric modulators bind to a site distinct from that where the receptor agonist would normally bind.

As used herein, “ivermectin” or “IVM” intends a mixture containing at least 90% 5-O demethyl-22,23-dihydroavermectin A₁ₐ and less than 10% 5-O-demethyl-25-de(1-methylpropyl)-22,23-dihydro-25-(1-methylethyl)avermectin A₁ₐ, generally referred to as 22,23-dihydroavermectin B₁ₐ and B_{1b}, or H₂B₁ₐ and H₂B_{1b}, respectively. The respective empirical formulas are C₄₈H₇₄O₁₄ and C₄₇H₇₂O₁₄, with molecular weights of 875.10 and 861.07, respectively. The structural formulas are:

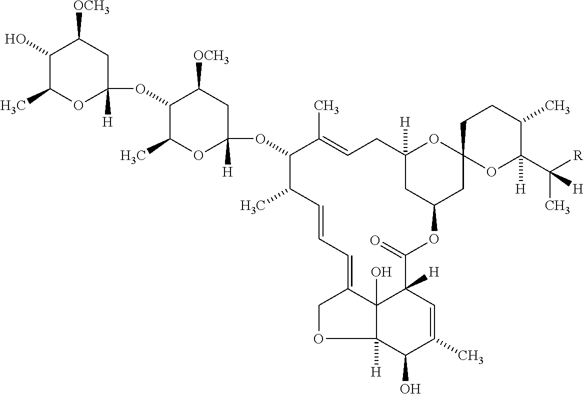

Regarding IVM, in component B₁ₐ, R═C₂H₅ and in component B_{1b}, R═CH₃. It is sold under the branded name Stromectol™ and has been used as an anti-parasitic. U.S. Pat. No. 4,853,372, issued Aug. 1, 1989 discloses a liquid formulation suitable for parenteral administration. U.S. Pat. No. 4,389,397 discloses a liquid formulation suitable for oral or parenteral administration.

The term “ivermectin analogue” refers to compounds having the ring structure of ivermectin. In this context, the requisite ring structure does not consider unsaturation or substituents on the rings. Ivermectin is a mixture of 5-O demethyl-22,23-dihydroavermectin A₁ₐ (at least 90%) and 5-O-demethyl-25-de(1-methylpropyl)-22,23-dihydro-25-(1-methylethyl)avermectin A₁ₐ (less than 10%). Ivermectin is also referred to as 22,23-dihydroavermectin B₁ₐ and B_{1b}, or H₂B₁ₐ and H₂B_{1b}, respectively. The present definition of ivermectin analogue also includes ivermectin.

An ivermectin analogue-containing composition comprises at least one ivermectin analogue having the following formula:

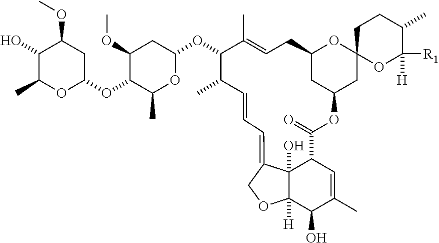

wherein R₁ is a C₁-C₁₀ alkyl. In a refinement, R₁ is non-cyclic C₁-C₁₀ alkyl. In another refinement, R₁ is methyl, ethyl, isopropyl, n-propyl, isobutyl, sec-butyl, or n-butyl. In still another refinement, R₁ is isopropyl, n-propyl, isobutyl, or sec-butyl. In a variation, the ivermectin analogue-containing composition comprises ivermectin.

“Alkyl” refers to monovalent saturated aliphatic hydrocarbyl groups having from 1 to 10 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

Ivermectin, which is formally an ivermectin analogue, is described by the following formula:

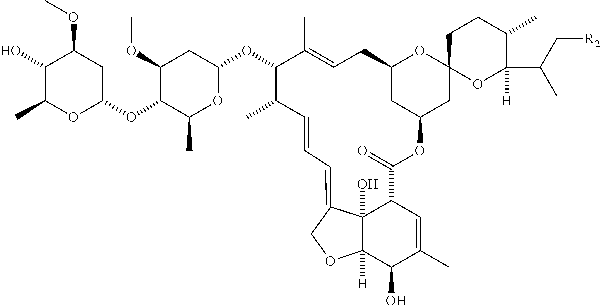

where for B₁ₐ, R₂ is methyl and for B_{1b}, R₂ is hydrogen. Ivermectin typically contains more than 90% B₁ₐ and less than 10% B_{1b}.

Another ivermectin analogue is abamectin (ABM) which is described by the following formula:

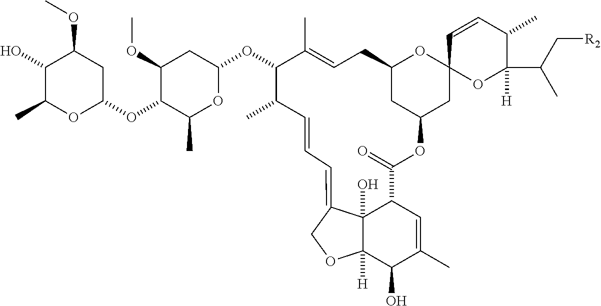

where for B₁ₐ, R₂ is methyl and for B_{1b}, R₂ is hydrogen. Abamectin typically contains more than 80% B₁ₐ and less than 20% B_{1b}.

Another ivermectin analogue is selamectin (SEL) which is described by the following formula:

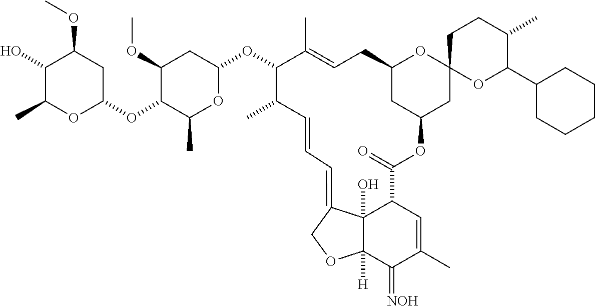

As used herein, the term “avermectin compounds” intends a series of 16-membered macrocyclic lactone derivatives. These naturally occurring compounds are generated as fermentation products by Streptomyces avermtilis. Eight different avermectins were isolated in four pairs of homologue compounds, with a major (a-component) and minor (b-component) component usually in ratios of 80:20 to 90:10.

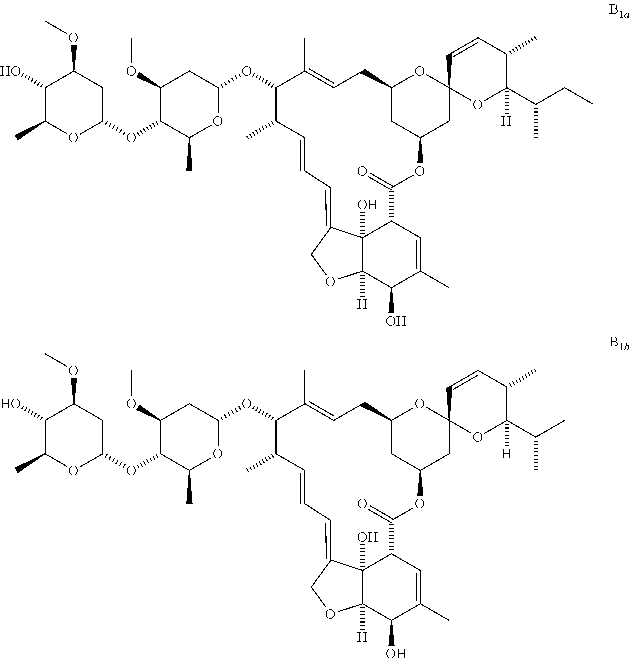

Other anthelmintic derived from the avermectins include ivermectin, selamectin, doramectin and abamectin. Methods to make the compounds and formulations containing the compounds are known in the art, e.g., U.S. Pat. Nos. 5,773,422; 5,583,029; and 8,791,153, and EP 0524687A1.

An example of an avermectin compound is moxidectin. The molecular formula is C₃₇H₅₃NO₈. It is available from a number of commercial vendors, e.g., ChemScene, Sigma Aldrich, Alurora Fine chemicals.

“L-dopamine therapy” intends an agent that increases dopamine in vivo. Non-limiting examples include L-DOPA and Sinemet™.

“L-DOPA” is also known as levodopa, intends the amino acid L-3,4-didhydrophenylalanine.

“Carbidopa” is an inhibitor of aromatic amino acid decarboxylation. It is a white, crystalline compound, slightly soluble in water, with a molecular weight of 244.3. It is designated chemically as N-amino-α-methyl-3-hydroxy-L-tyrosine monohydrate. Its empirical formula is C₁₀H₁₄N₂O₄.H₂O. It is sold under the tradename Lodysn™.

“Benserazide” is also called Serazide or Ro4-4602, and is peripherally acting aromatic L-amino acid decarboxylase or DOPA decarboxylase inhibitor. It is used in the management of PD and in combination with L-DOPA, as co-beneidopa, under the tradename Madopar™, by Roche.

The combination of levodopa and carbidopa is sold under the brand names Kinson™, Sinemet™, Pharmocopa™ and Atamet™.

“Sinemet™” is a combination of carbidopa and levodopa. Sinemet™ is supplied as tablets in three strengths: SINEMET 25-100, containing 25 mg of carbidopa and 100 mg of levodopa. SINEMET 10-100, containing 10 mg of carbidopa and 100 mg of levodopa. SINEMET 25-250, containing 25 mg of carbidopa and 250 mg of levodopa.

“Pharmaceutically acceptable salt” refers to salts of a compound, which salts are suitable for pharmaceutical use and are derived from a variety of organic and inorganic counter ions well known in the art and include, when the compound contains an acidic functionality, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate (see Stahl and Wermuth, eds., “Handbook of Pharmaceutically Acceptable Salts,” (2002), Verlag Helvetica Chimica Acta, Zurich, Switzerland), for a discussion of pharmaceutical salts, their selection, preparation, and use.

Generally, pharmaceutically acceptable salts are those salts that retain substantially one or more of the desired pharmacological activities of the parent compound and which are suitable for in vivo administration. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (e.g., an alkali metal ion, an alkaline earth metal ion, or an aluminum ion) or by an ammonium ion (e.g., an ammonium ion derived from an organic base, such as, ethanolamine, diethanolamine, triethanolamine, morpholine, piperidine, dimethylamine, diethylamine, triethylamine, and ammonia).

A solvate of a compound is a solid-form of a compound that crystallizes with less than one, one or more than one molecules of a solvent inside in the crystal lattice. A few examples of solvents that can be used to create solvates, such as pharmaceutically acceptable solvates, include, but are not limited to, water, C₁-C₆ alcohols (such as methanol, ethanol, isopropanol, butanol, and can be optionally substituted) in general, tetrahydrofuran, acetone, ethylene glycol, propylene glycol, acetic acid, formic acid, and solvent mixtures thereof. Other such biocompatible solvents which may aid in making a pharmaceutically acceptable solvate are well known in the art. Additionally, various organic and inorganic acids and bases can be added to create a desired solvate. Such acids and bases are known in the art. When the solvent is water, the solvate can be referred to as a hydrate. In some embodiments, one molecule of a compound can form a solvate with from 0.1 to 5 molecules of a solvent, such as 0.5 molecules of a solvent (hemisolvate, such as hemihydrate), one molecule of a solvent (monosolvate, such as monohydrate) and 2 molecules of a solvent (disolvate, such as dihydrate).

An animal, subject or patient for diagnosis or treatment refers to an animal such as a mammal, or a human, ovine, bovine, feline, canine, equine, simian, etc. Non-human animals subject to diagnosis or treatment include, for example, simians, murine, such as, rat, mice, canine, leporid, livestock, sport animals, and pets. In one aspect, the subject is a human. It is to be understood that the terms “subject” and “patient” are interchangeable.

A “composition” as used herein, refers to an active agent, such as an agent as disclosed herein and a carrier, inert or active. The carrier can be, without limitation, solid such as a bead or resin, or liquid, such as phosphate buffered saline.

“Administration,” “administering” and the like intends by any appropriate means, e.g., intravenously, orally, by suppository, inhalation, or other, an agent, composition or combination as described herein.

Administration or treatment in “combination” refers to administering two agents such that their pharmacological effects are manifest at the same time. Combination does not require administration at the same time or substantially the same time, although combination can include such administrations.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response such as treatment of PD or PD symptoms.

As used herein, “treating” or “treatment” of a disease in a patient refers to (1) preventing the symptoms or disease from occurring in an animal that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein “conditions for binding of the candidate agent to the P2X4 receptor” can be found throughout the examples.

### Therapeutic Methods

Provided herein are methods for the treatment of one or more of: Parkinson's disease (PD); PD symptoms; movement disorders; and neurodegenerative diseases linked to changes in dopamine, dopamine signaling, or dopamine expression, in a subject in need thereof. The methods comprise, or alternatively consist essentially of, or yet further consist of, administering to the subject an effective amount of an agent that potentiates dopaminergic neurotransmission. The methods as disclosed herein are not limited by the mode of administration, and include without limitation, orally, intravenously or by suppository. A subject in need thereof includes mammals, such as pets and veterinary animals, and human patients. As used herein, an effective amount intends an amount to reduce or alleviate the symptoms of the disease or disorder, and vary with the subject being treated, the age, the disease or disorder, and the treating physician. The treating physician or her assistant can determine when the method has been successful by observing symptoms. In one aspect, the physician can measure the amount of dopamine in the patient using a test, e.g., a catecholamine blood test or a urine test for dopamine levels.

In some refinements, the effective amount is between about 0.05 mg/kg to about 20 mg/kg. In some refinements, the effective amount is between about 0.1 mg/kg to about 10 mg/kg. In some refinements, the effective amount is between about 0.2 mg/kg to about 8 mg/kg. In some refinements, the effective amount is between about 0.5 mg/kg to about 6 mg/kg. In some refinements, the effective amount is between about 1 mg/kg to about 5 mg/kg. In some refinements, the effective amount is between about 2 mg/kg to about 5 mg/kg. In some refinements, the effective amount is 2.5 mg/kg. In some refinements, the effective amount is 5 mg/kg.

In some refinements, the effective amount is administered once a day. In some refinements, the effective amount is administered twice a day. In some refinements, the effective amount is administered thrice a day. In some refinements, the effective amount is administered four times a day. In some refinements, the effective amount is administered continuously through infusion. In further embodiments, the effective amount is administered in the same or different dosage amount, and/or in the same or different routes of administration, and/or in the same or different formulation.

In some refinements, the effective amount is administered daily for about 2 days to about 7 days. In some refinements, the effective amount is administered daily for about 2 days to about 2 weeks. In some refinements, the effective amount is administered daily for about 1 week to about 1 month. In some refinements, the effective amount is administered daily for about 1 month to about 6 months. In some refinements, the effective amount is administered daily for about 6 months to about 1 year. In some refinements, the effective amount is administered daily for about 1 year to about 2 years. In some refinements, the effective amount is administered daily for about 2 years to about 5 years. In some refinements, the effective amount is administered daily for the lifetime of the subject. In further embodiments, the effective amount is administered in the same or different dosage amount, and/or in the same or different routes of administration, and/or in the same or different formulation.

Purinergic P2X4 receptors (P2X4Rs) belong to the P2X superfamily of ion channels regulated by ATP. Applicant had demonstrated that P2X4R knockout (KO) mice exhibited deficits in sensorimotor gating, social interaction, and ethanol drinking behavior. Dopamine (DA) dysfunction may underlie these behavioral changes, but there is no direct evidence for P2X4Rs' role in DA neurotransmission. To test this hypothesis, Applicant measured markers of DA function and dependent behaviors in P2X4R KO mice. P2X4R KO mice exhibited altered density of pre-synaptic markers including tyrosine hydroxylase, dopamine transporter; post-synaptic markers including dopamine receptors and phosphorylation of downstream targets including dopamine and cyclic-AMP regulated phosphoprotein of 32 kDa and cyclic-AMP-response element binding protein in different parts of the striatum. Ivermectin, an allosteric modulator of P2X4Rs, significantly affected dopamine and cyclicAMP regulated phosphoprotein of 32 kDa and extracellular regulated kinasel/2 phosphorylation in the striatum. Sensorimotor gating deficits in P2X4R KO mice were rescued by DA antagonists. Using the 6-hydroxydopamine model of DA depletion, P2X4R KO mice exhibited an attenuated levodopa (L-DOPA)-induced motor behavior, whereas ivermectin enhanced this behavior. Collectively, these findings identified an important role for P2X4Rs in maintaining DA homeostasis and illustrate how this association is important for CNS functions including motor control and sensorimotor gating.

Non-limiting examples of PD symptoms are described herein, e.g., tremors, rigidity and bradykinesia. In another aspect, the neurodegenerative diseases linked to changes in dopamine are selected from the group of: encephalitis, concussions, dopamine-responsive dystonia, and Parkinsonian syndrome. As used herein, “a subject in need thereof” intends a subject displaying symptoms indicative of PD and include, but are not limited to any of the above symptoms unless specifically referenced.

Agents useful in the methods include ivermectin (IVM), an IVM analogue, a P2X4R allosteric modulator, and avermectin compounds as disclosed herein, as well as pharmaceutically acceptable salts and solvates thereof, where appropriate.

In some embodiments, the agent that potentiates dopaminergic neurotransmission includes IVM and/or moxidectin (MOX), or a pharmaceutically acceptable salt or solvate of each thereof. In some embodiments, the agent that potentiates dopaminergic neurotransmission includes IVM or a pharmaceutically acceptable salt or solvate thereof. In some embodiments, the agent that potentiates dopaminergic neurotransmission includes MOX or a pharmaceutically acceptable salt or solvate thereof. In some embodiment, the method further comprises analyzing the concentration of dopamine or DOPAC in the subject using an analytical method, for example, gas chromatography mass spectrometry (GCMS).

A non-limiting example of an IVM analogue is a compound of the formula:

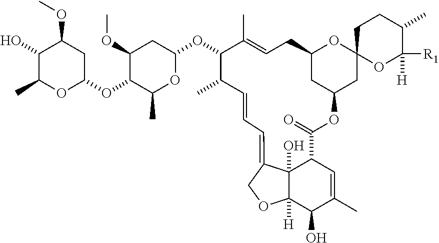

wherein R₁ is a C₁-C₁₀ alkyl. In one aspect, R₁ is methyl, ethyl, isopropyl, n-propyl, isobutyl, sec-butyl, or n-butyl. In another aspect, R₁ is isopropyl, n-propyl, isobutyl, or sec-butyl. In a further aspect, the IVM analogue-containing composition comprises, or consists essentially of, or yet further consists of, IVM.

In another aspect, the method further comprises administering to the subject an effective amount of L-dopamine therapy. The L-dopamine therapy is administered prior to, subsequent to, or concurrently with, the agent that potentiates dopaminergic neurotransmission. Non-limiting examples of L-dopamine therapies, include without limitation an agent comprising L-DOPA also known as levodopa, that can be further modified by administering an effective amount of carbidopa or benserazide. In some embodiments, the L-DOPA and carbidopa are administered in a mass ratio of 4 to 1 or 10 to 1. Such a combination therapy can be administered by administration of an effective amount of Sinemet™. As used herein, an effective amount intends an amount to reduce or alleviate the symptoms of the disease or disorder, and vary with the subject being treated, the age, the disease or disorder, and the treating physician. The treating physician or her assistant can determine when the method has been successful by observing symptoms. In one aspect, the physician can measure the amount of dopamine in the patient using a test, e.g., a catecholamine blood test or a urine test for dopamine levels. In some embodiments, the subject is a mammal. In a further embodiment, the subject is a human.

In some refinements, the effective amount of L-dopamine therapy is between about 0.05 mg/kg to about 50 mg/kg. In some refinements, the effective amount of L-dopamine therapy is between about 0.1 mg/kg to about 20 mg/kg. In some refinements, the effective amount of L-dopamine therapy is between about 0.2 mg/kg to about 10 mg/kg. In some refinements, the effective amount of L-dopamine therapy is between about 0.5 mg/kg to about 6 mg/kg. In some refinements, the effective amount of L-dopamine therapy is between about 1 mg/kg to about 5 mg/kg. In some refinements, the effective amount of L-dopamine therapy is between about 2 mg/kg to about 5 mg/kg. In some refinements, the effective amount of L-dopamine therapy is 2.5 mg/kg. In some refinements, the effective amount of L-dopamine therapy is 5 mg/kg.

### Methods of Selecting a Candidate Agent for Treatment

Also provided is a method for selecting a candidate agent for the treatment of one or more of: Parkinson's disease (PD); PD symptoms; and neurodegenerative diseases linked to changes in dopamine, dopamine signaling, or dopamine expression, in a subject in need thereof, the method comprising contacting a compound with a cell expressing a P2X4 receptor under conditions for binding of the candidate agent to the P2X4 receptor and selecting the compound as the candidate agent if it potentiates P2X4 receptor activity. In some embodiments, the cell is a neural cell. In some embodiments, the cell is modified to express a P2X4 receptor. It is to be understood that “compound” as it recited in this method may refer to, but not be limited by, a small molecule, a macromolecule, or a biologic.

### Compositions

Compositions, including pharmaceutical compositions comprising the compounds described herein can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries which facilitate processing of the compounds provided herein into preparations which can be used pharmaceutically.

The compounds of the technology can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration.

In one embodiment, this disclosure relates to a composition comprising a compound as described herein and a carrier.

In another embodiment, this disclosure relates to a pharmaceutical composition comprising a compound as described herein and a pharmaceutically acceptable carrier.

In another embodiment, this disclosure relates to a pharmaceutical composition comprising a therapeutically effective amount of a compound as described herein and a pharmaceutically acceptable carrier.

The pharmaceutical compositions for the administration of the compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition the compound provided herein is included in an amount sufficient to produce the desired therapeutic effect. For example, pharmaceutical compositions of this disclosure may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g., starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

### Use of Compounds for Preparing Medicaments

The compounds and compositions of the present invention are also useful in the preparation of medicaments to treat a variety of pathologies as described herein. The methods and techniques for preparing medicaments of a composition are known in the art. For the purpose of illustration only, pharmaceutical formulations and routes of delivery are detailed herein.

Thus, one of skill in the art would readily appreciate that any one or more of the compositions described above, including the many specific embodiments, can be used by applying standard pharmaceutical manufacturing procedures to prepare medicaments to treat the many disorders described herein. Such medicaments can be delivered to the subject by using delivery methods known in the pharmaceutical arts.

### Administration of Additional Therapeutic Agents

The methods disclosed herein can further comprise, or alternatively consist essentially of, or yet further consist of administration of an effective amount of additional therapeutic agents to augment or enhance the therapeutic efficacy of the disclosed methods. Non-limiting examples of additional therapeutic agents to augment or enhance the therapeutic efficacy of the disclosed methods include rasagiline, pramipexole, ropinirole, amantadine, selegiline, entacapone, rotigotine, benztropine, biperiden, trihexyphenidyl, apomorphine, rivastigmine, bromocriptine, rotigotine, trihexane, belladonna, safinamide, and tolcapone.

### EXPERIMENTAL EXAMPLES

### Example 1: Linking P2XR4 Function to DA Signaling Via Administration of IVM to P2XR4 WT and P2XR4 KO Mice

### Materials and Methods

Purinergic P2X receptors are cation permeable ion channels gated by ATP. They form homo- and heterotrimeric channels from seven subunits, namely, P2X1-P2X7 (North, R. A. (2002) Physiol. Rev. 82:1013-1067; Khakh, B. S. et al. (2012) Neuron 76:51-69). Among them, P2X4 receptors (P2X4Rs) are abundantly expressed on neurons and glial (microglia and astrocytes) cells across the CNS as well as the PNS (Le, T. K. et al. (1998) Neuroscience 83:177-190). Gene knockout and pharmacological strategies have implicated P2X4R mediated transmission in hippocampal synaptic plasticity, inflammatory processes in the spinal cord, and neuroendocrine functions (Sim, J. A. et al. (2006) J. Neurosci. 26:9006-9009; Ulmann, L. et al. (2008) J. Neurosci. 28:11263-11268; Zemkova, H. et al. (2010) Am. J. Physiol. Endocrinol. Metab. 298:E644-E651).

It has been reported that mice deficient in the p2rx4 gene [i.e., P2X4R knockout (KO)] exhibited deficits in sensorimotor gating, social behavior, and ethanol drinking behavior (Wyatt, L. R. et al. (2013) Neuropsychopharmacology 38:1993-2002; Wyatt, L. R. et al. (2014) Neurochem. Res. 39:1127-1139). However, there was no identification of any molecular mechanism that could explain these behavioral deficits. Without being bound by theory, Applicant hypothesizes that one plausible mechanism could be a result of P2X4Rs modulating major neurotransmitter systems including the glutamate and GABA systems. For instance, P2X4Rs are suggested to regulate post-synaptic currents mediated by NMDA receptors, a-amino-3-hydroxy-5-methylisoxazole-4-propionate Receptors, and GABAA receptors as well as pre-synaptic release of glutamate and GABA (Gu, J. G. et al. (1997) Nature 389:749-753; Hugel, S. et al. (2002) J. Neurosci. 20:2121-2130; Andries, M. et al. (2007) Neurobiol. Dis. 25:8-16; Baxter, A. W. et al. (2011) Eur. J. Neurosci. 34:213-220; Jo, Y. H. et al. (2011) J. Biol. Chem. 286:19993-20004). Moreover, P2X4R KO mice exhibited altered subunit expression of multiple glutamatergic and GABAA receptors across multiple brain regions. This latter finding suggests that P2X4R deficiency disrupts homeostasis of post-synaptic ionotropic receptors (Wyatt, L. R. et al. (2013) Neuropsychopharmacology 38:1993-2002; Wyatt, L. R. et al. (2014) Neurochem. Res. 39:1127-1139). Notably, disruption of glutamatergic and GABAergic function has been linked to deficits in sensorimotor gating, social interaction, and ethanol drinking behavior (Blednov, Y. A. (2003) J. Pharmacol. Exp. Ther. 305:854-863; Duncan, G. E. et al. (2004) Behay. Brain Res. 153:507-519; Du, X. et al. (2012) Neurotoxicol. Teratol. 34:177-186). Together, these findings support the hypothesis that P2X4Rs can interact with other ionotropic receptors in regulation of multiple CNS functions.

In contrast to the building evidence supporting a role for P2X4Rs in glutamatergic and GABAergic function, little is known regarding the interaction of P2X4Rs with dopamine (DA) neurotransmission. Early evidence suggests that P2X4Rs are indirectly involved in DA neurotransmission (Krugel, U. et al. (2001) Neuropharmacology 40:1084-1093; Krügel, U. et al. (2003) Synapse 47:134-142; Xiao, C. et al. (2008) J. Pharmacol. Exp. Ther. 327:196-205), but the direct role for P2X4Rs in regulating DA homeostasis has not been demonstrated. Considering that P2X4Rs are expressed on DA neurons and GABAergic medium spiny neurons (MSNs) of the basal ganglia (Amadio, S. et al. (2007) Purinergic Signal. 3:389-398; Heine, C. et al. (2007) Neuroscience 149:165-181) and the behavioral deficits exhibited by P2X4R KO mice may represent DA dysfunction (Zhou, F. C. et al. (1995) Alcohol 12:403-412; Gendreau, P. L. et al. (2000) Behay. Brain Res. 114:107-117; Ralph, R. J. et al. (2001) J. Neurosci. 21:305-313; Rodriguiz, R. M. et al. (2004) Behay. Brain Res. 148:185-198), Applicant hypothesized that P2X4Rs control DA signaling with a relevant impact on DA-associated behaviors.

Applicant reports herein that using a P2X4R KO mouse model as a genetic approach and ivermectin (IVM), a positive allosteric modulator of P2X4Rs (Khakh, B. S. et al. (1999) J. Neurosci. 19:7289-7299; Priel, A. et al. (2004) J. Gen. Physiol. 123:281-293; Jelinkova, I. et al. (2006) Biochem. Biophys. Res. Commun. 349:619-625; Jelinkova, I. et al. (2008) Pflugers Arch. 456:939-950; Hattori, M. et al. (2012) Nature 485:207-212), as a pharmacological approach could be used to test the aforementioned hypothesis. Protein densities of different markers of DA neurotransmission including tyrosine hydroxylase (TH), dopamine transporter (DAT), dopamine D1 and D2 receptors (D1Rs and D2Rs) were measured, and downstream targets integral to DA signaling including dopamine and cyclic-AMP regulated phosphoprotein of 32 kDa (DARPP-32), extracellular regulated kinase-1/2 (ERK 1/2), and cyclic-AMP-response element binding protein (CREB) in different regions of the striatum of P2X4R KO and wildtype (WT) male mice. Applicant also measured the degree of phosphorylation of DARPP-32, ERK 1/2, and CREB isolated from different striatal regions of WT and P2X4R KO mice in the presence and/or absence of IVM. The interaction between P2X4Rs and DA system in the regulation of CNS functions was addressed by employing behavioral pharmacology paradigms. The 6-Hydroxydopamine model (6-OHDA) of DA depletion was used to link P2X4R function with DA neurotransmission in modulation of motor control. Finally, using the prepulse inhibition (PPI) of acoustic startle reflex coupled with DA antagonists, Applicant evaluated the effects of DA dysregulation as it is pertained to sensorimotor gating deficits. Overall, the findings support the hypothesis that P2X4R function plays a role in maintaining DA signaling with an impact on DA-associated behaviors such as motor control and sensorimotor gating.

Animals

Experimentally naive male WT and P2X4R KO mice were obtained from Applicant's breeding colony at the University of Southern California. The breeding colony was established from a previous P2X4R KO colony that was maintained on a C57BL/6 background (Sim, J. A. et al. (2006) J. Neurosci. 26:9006-9009). The overall breeding scheme for generation of P2X4R KO mice and genotyping has been described previously (Wyatt, L. R. et al. (2013) Neuropsychopharmacology 38:1993-2002; Wyatt, L. R. et al. (2014) Neurochem. Res. 39:1127-1139). Mice were housed in groups of 5 per cage in rooms maintained at 22° C. with 12/12 h light: dark cycle and ad libitum access to food and water. All experiments were undertaken in compliance to guidelines established by National Institute of Health (NIH) and approved by the Institutional Animal Care and Use Committee of University of Southern California.

Two- to three-month-old P2X4R KO and WT mice were used for biochemical assays. Two- to four-month-old P2X4R KO and WT mice were used for the motor behavior studies. Four- to six-month-old mice were used for the PPI of acoustic startle reflex study with DA antagonists.

### Materials

Levodopa (L-DOPA) (Sinemet; 100 mg L-DOPA, 25 mg Carbidopa per pill) was diluted in 0.9% saline solution to achieve a concentration of 0.75 mg/mL. IVM (Norbrook, Lenexa, Kans., USA) was diluted in 0.9% saline solution, to achieve a concentration of 0.5 mg/mL and injected at a volume of 0.01 mL/g of body weight. Propylene glycol (Sigma-Aldrich, St. Louis, Mo., USA) was used as the vehicle control for IVM. SCH-23390 HCl and raclopride tartrate (Sigma-Aldrich) were dissolved in 0.9% saline at concentrations of 0.2 and 0.6 mg/mL, respectively. Both drugs were injected at a volume of 0.005 mL/g of body weight.

### Western Immunoblotting

### Tissue Preparation

The dorsal and ventral striata were dissected from P2X4R KO and WT mice following killing with CO₂ asphyxiation. For the experiment that tested the effects of IVM (5 mg/kg, i.p.) on dopaminergic signaling, the dorsal and ventral striatal regions were dissected out 8 h post-drug administration. The dorsal and ventral striata were dissected out as per the neuroanatomical landmarks described in the mouse brain atlas (Franklin, B. J. and Paxinos, G. (2007) The Mouse Brain in Stereotaxic Coordinates. Elsevier Academic Press, Amsterdam). The brain tissues were homogenized in a buffer containing 50 mM Tris-HCl pH (8.0), 150 mM NaCl, 1 mM EDTA, 0.1% sodium dodecyl sulfate, 1/100 dilution proteinases inhibitor cocktail (Millipore, Temecula, Calif., USA), and protein content was determined using bicinchoninic acid assay kit (Thermo Scientific, Rockford, Ill., USA). Homogenates were treated with a cocktail of phosphatase inhibitors (1 mM sodium pyrophosphate, 10 mM sodium fluoride, 0.5 mM sodium orthovandate, 10 mM β-glycerol phosphate, 1 μM microcysteine LR) (Sigma-Aldrich) for detection of phosphoproteins.

### Immunoblotting Procedure

Protein samples of 50 lg ran on 10% sodium dodecyl sulfate-polyacrylamide gel electrophoresis gels and transferred onto polyvinylidine diflouride membranes using semi-dry transfer method (Trans turbo blot; BioRad Laboratories, Hercules, Calif., USA). Striatal samples from WT and P2X4R KO mice were made to run on the same gel and transferred onto the same membrane. Nonspecific binding was blocked by incubation in 5% non-fat dry milk, followed by incubation with primary antibodies overnight at 4° C. The antibodies used were rabbit anti-TH (1:5000), mouse anti-DAT (1:1000), rabbit anti-D2 receptor (1:1000), rabbit anti-DARPP-32 (1:1000) (Millipore), rabbit anti-D1 receptor (1:500; SantaCruz biotechnology, Santacruz, Calif., USA), mouse anti-total ERK 1/2 (1:1000), rabbit anti-total CREB (1:1000) (Cell Signaling Technology, Beverly, Mass., USA), mouse anti-β-actin (1:20 000; Sigma-Aldrich), and mouse anti-α-tubulin (1:10 000; Millipore). The antibodies for phospho proteins included rabbit antiphospho-Thr34-DARPP-32 (1:400; Millipore), rabbit anti-phospho-Ser133-CREB (1:500), rabbit anti-diphospho-Thr202/Tyr204-ERK 1/2 (1:500) (Cell Signaling Technology). Secondary antibodies included goat anti-mouse and goat anti-rabbit antibodies (1:10 000; BioRad Laboratories). Bands were visualized using chemiluminescence method (Clarity western plus enhanced chemiluminescence substrate; BioRad Laboratories) followed by exposure to HyBlot autoradiography films (Denville Scientific, Metuchen, N.J., USA). Protein quantification was carried out by optical densitometry using ImageJ software (NIH, Bethesda, Md., USA). Protein densities were normalized to β-actin or α-tubulin levels.

### HPLC Assay

Brain tissue was homogenized with 0.5 M perchloric acid, centrifuged at 16 873 g for 12 min at 4° C., and protein was resuspended in 0.5 M NaOH. Protein content was detected by bicinchoninic acid assay. DA concentrations were determined using electrochemical detection method consisting a ESA model Coularray 5600A coupled with a four channel analytical cell at 175, 50, 220, and 300 mV. Samples were injected with ESA autosampler (Chelmsford, Mass., USA) and DA was separated by a 150×3.2 mm reverse phase 3 μm diameter C-18 column regulated at 28° C. The mobile phase MD-TM (ESA) consisted of acetonitrile in phosphate buffer and a non-ion-pairing reagent delivered at a rate of 0.6 mL/min. The HPLC was integrated with a Dell GX 280 computer with analytical programs including ESA Coularray for Windows software.

### Acoustic Startle Reflex and PPI of Startle

### Apparatus

Acoustic startle reflex and PPI were tested as previously described (Wyatt, L. R. et al. (2013) Neuropsychopharmacology 38:1993-2002). The apparatus used for detection of startle reflex (San Diego Instruments, San Diego, Calif., USA) consisted of a standard cage placed in sound attenuated chambers with fan ventilation. Each cage consisted of a Plexiglass cylinder of 3 cm diameter, mounted on piezoelectric accelerometric platform connected to an analog-digital converter. Background noise and acoustic bursts were conveyed by two separate speakers, each one oriented appropriately so as to produce a variation in sound within 1 dB across the startle cage. Both speakers and startle cages were connected to the main PC, which detected and analyzed all chamber variables with Startle software (San Diego Instruments). Before each testing session, acoustic stimuli were calibrated via a digital sound level meter.

### Startle and PPI Session

During the baseline session, mice were exposed to background noise of 70 dB and, after an acclimatization period of 5 min, were presented with 12 40 ms trials of 115 dB interposed with three trials of a 82 dB pre-stimulus preceding the 115 dB by 100 ms. Subsequently, treatment groups were established so that the average startle response and % PPI were equivalent within the WT and P2X4R KO groups. On the testing day, each mouse was placed in the cage and exposed to a 5 min acclimatization period with a 70 dB white noise background, which continued for remainder of the session. Each session consisted of three consecutive sequences of trials. Unlike the first and third session, wherein the mice were exposed to five alone pulse trials of 115 dB, the second period consisted of a pseudorandom sequence of 50 trials, including 12 pulse alone trials, 30 trials of pulse preceded by 73 dB, 76 dBor 82 dBprepulse (respectively, defined as PPI 3, PPI 6, and PPI 12; 10 for each level of prepulse loudness), and 8 no stimulus trials, wherein the mice were presented with background noise without any prepulse or pulse stimuli. Intertrial intervals were randomly chosen between 10 and 15 s. Delta PPI (APPI) was calculated as mean startle amplitude for pulse alone trials—(mean startle amplitude for prepulse trial). DA antagonists, SCH-23390 (1 mg/kg, i.p) and raclopride (3 mg/kg, i.p) were administered 10 min prior to testing session.

### 6-OHDA Lesioning in Mice and Motor Behavior Testing

### 6-OHDA Lesioning Surgery

Mice were treated with desipramine hydrochloride (25 mg/kg, i.p.) (Sigma-Aldrich) 30 min prior to surgery to prevent concurrent damage of noradrenergic pathways by 6-OHDA. Mice were anesthetized with avertin (25 mg/kg, i.p.) and placed in the stereotaxic apparatus. Two μL of freshly prepared 6-OHDA bromide salt (4 mg/mL in 0.2% ascorbic acid and 0.9% saline; Sigma-Aldrich) was unilaterally infused into left median forebrain bundle (from Bregma point: 1.2 mm posterior, 1.1 mm lateral, 5 mm ventral) (Franklin, B. J. and Paxinos, G. (2007) The Mouse Brain in Stereotaxic Coordinates. Elsevier Academic Press, Amsterdam) at a rate of 0.5 μL/min using a 10 μL Hamilton syringe and a microlitre syringe pump. The injection cannula was left in place for 3-5 min to prevent reflux and ensure complete absorption. Post-operative procedures involved daily subcutaneous (s.c.) injections with sucrose (5% w/v in saline) and warming on a heating pad for 2 weeks.

### Motor Behavior Testing

Mice were subjected to behavioral testing 3 weeks after 6-OHDA lesioning surgery. Mice were placed in a plastic cylinder (318 mm in diameter) with a video camera mounted above it. Baseline rotational behavior was established by saline injections which causes the mouse to rotate toward the lesioned side with lesser DA activity (ipsilateral rotations). L-DOPA (5 mg/kg, s.c.) was administered 5 min prior to behavioral testing and the number of contralateral and ipsilateral rotations were counted in every 10-minute interval for a total period of 90 min. After monitoring the behavioral activity with L-DOPA alone, the same cohort of WT and P2X4R KO mice received a combination of IVM (5 mg/kg, i.p.) and L-DOPA (5 mg/kg, s.c.) to study the modulatory effect of IVM on L-DOPA-induced motor behavior. IVM and L-DOPA were administered 8 h and 5 min, respectively, prior to behavioral testing. To monitor effect of IVM alone on rotational behavior, a separate cohort of 6-OHDA lesioned WT mice received IVM (5 mg/kg, i.p) 8 h prior to behavioral testing and motor activity was monitored for 2 h. The 8 h time point was selected as IVM has been shown to achieve maximal concentration in brain and plasma post 8 h (Yardley, M. et al. (2012) Neuropharmacology 63:190-201).

### Peroxidase-Based Immunohistochemistry

### Collection and Processing of Brain Tissues

Transcardial perfusion was performed using 0.9% NaCl/4% phosphate-buffered paraformaldehyde. Perfused brains were post-fixed overnight followed by storage in 20% sucrose for 48 h and quickly frozen with 4-methylbutane on dry ice. Perfused brains were cut coronally at 25 μm thickness in a cryostat and stored in a cryoprotective solution containing 30% sucrose in phosphate buffered saline at 4° C. until further use.

### TH Staining Using Peroxidase Method of Immunohistochemistry in Ventral Mesencephelon

Brain sections were heated in 10 mM sodium citrate buffer (pH 8.5) for 30 min at 80° C. followed by permeabilization in Tris-buffered saline (TBS)+0.2% Triton-X for 30 min. Slices were then quenched in solution containing 10% methanol and 3% H₂O₂ in TBS for 10 min and subsequently blocked in 5% non-fat dry milk and in 5% normal goat serum (each step for 30 min). Sections were incubated overnight with rabbit anti-TH (1:5000) (Millipore) diluted in TBS+0.2% Triton-X containing 1% normal goat serum. The avidin-biotin complex method of detection was used, wherein slices were incubated with biotinylated goat anti-rabbit antibody (1:500) for 1 h and then with 1% avidin linked peroxidase complex for 45 min (Vector Laboratories, Burlingame, Calif., USA). This was followed by treatment with solution containing 0.05% 3,3-diaminobenzidine and 0.015% H₂O₂ in phosphate-buffered saline for 5 min, dehydration of slides in a dilution series of ethanol, clearance in xylene, and evaluation under light microscope.

### Statistical Analyses

For the western blotting analysis, the average of densitometry values of WT striatal samples was used to arbitrarily normalize WT samples to 1 and the P2X4R KO striatal samples were normalized by dividing each densitometry value by the average of WT samples. Normalization of the two genotypes was done within the same membrane and presented as fold change of P2X4R KO versus WT in that membrane. For D1, D2, DARPP-32, ERK 1/2, and CREB immunoblotting; WT and P2X4R KO mice were generated in separate cohorts. The normalization of P2X4R KO to WT samples was done within the same cohort followed by combining data from two cohorts for analyses. The same method of normalization was used for the study that tested the effects of IVM on dopaminergic signaling in the dorsal and ventral striatum of WT and P2X4R KO mice. The average of vehicle-treated WT mice was used to arbitrarily normalize the WT control samples to 1 and the IVM treated WT, vehicle- and IVM-treated P2X4R KO mice were normalized by dividing each value by the average of vehicle-treated WT mice. Normalized density of proteins was expressed as mean±SEM. Phosphorylation was calculated as ratio of normalized values of phosphorylated form to total form of protein. DA levels were expressed as ng/mg of protein content. Unpaired Student t-test was used for analyzing the differences in protein densities and DA levels between WT and P2X4R KO groups. Two-way ANOVA with Bonferroni post hoc test was used to evaluate the effect of IVM on phosphorylation of various signaling molecules between WT and P2X4R KO. Pharmacological studies for motor behavior and sensorimotor gating were analyzed by two-way repeated measures ANOVA with time/PPI intensity and genotype/treatment as within and between-subjects variability, respectively, followed by Bonferroni post hoc test for multiple comparisons. Significance was set at p<0.05. All data were analyzed using GraphPad Prism software (San Diego, Calif., USA).

### Results

### P2X4R KO Mice Exhibit Alterations in Expression of Presynaptic DA Markers in Striatum

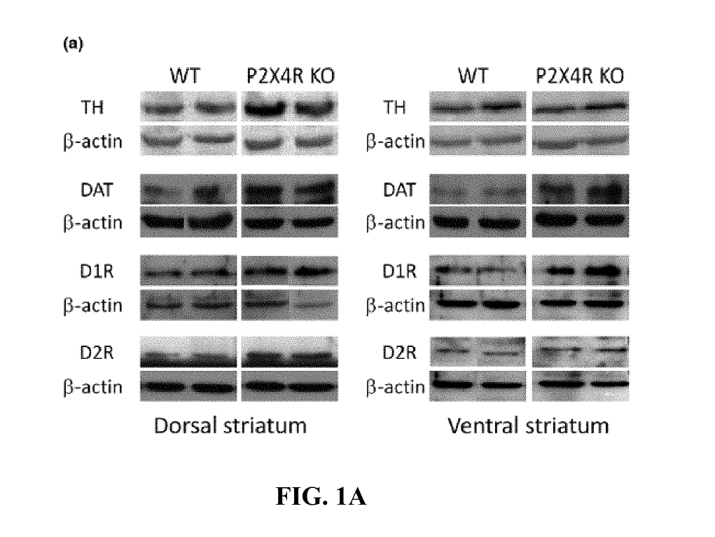

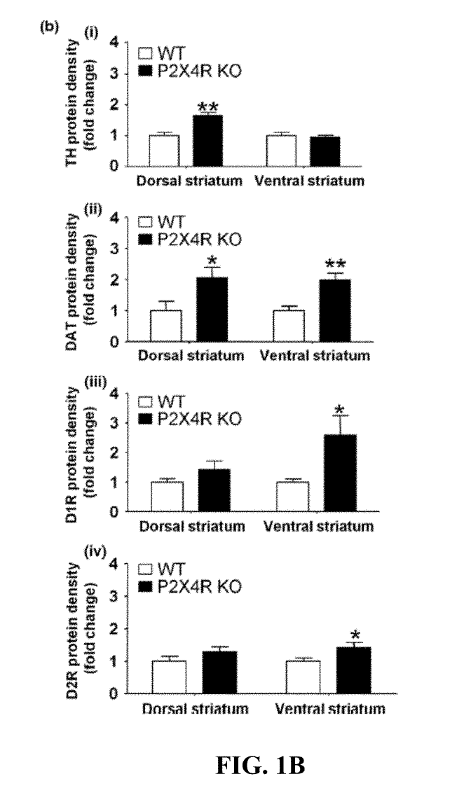

To investigate changes in pre-synaptic markers of DA neurotransmission, Applicant compared TH and DAT protein density between P2X4R KO and WT mice using western immunoblotting. P2X4R KO mice exhibited a significant increase in TH protein density in the dorsal striatum by 64% (p<0.01) but no change in protein density in the ventral striatum [FIG. 1A and FIG. 1B(i)]. P2X4R KO mice exhibited significant increases in DAT protein density by 106% (p<0.05) and by 98% (p<0.01) in the dorsal and ventral striatum, respectively [FIG. 1A and FIG. 1B(ii)].

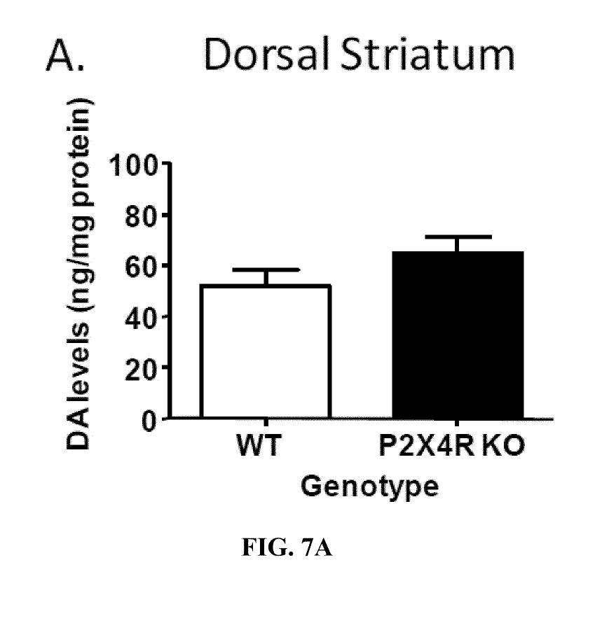

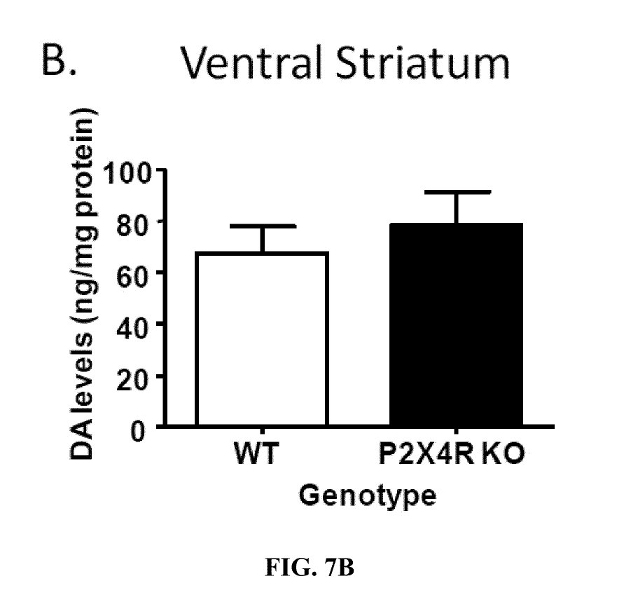

The significant increase in TH protein density in the dorsal striatum did not appear to be associated with an increase in DA levels as there were no significant changes in DA levels in the dorsal striatum (FIG. 7A). Moreover, there were no changes in DA levels in the ventral striatum (FIG. 7B) between P2X4R KO and WT mice.

### P2X4R KO Mice Exhibit Significant Alterations in Expression of DA Receptors and Downstream Targets

Based on the significant changes in TH and DAT protein densities, Applicant posits that changes within pre-synaptic DA environment could impact the densities of DA receptors and downstream targets. Among the DA receptor subtypes, D1Rs belong to excitatory Ga_{s/olf} family of G-coupled protein receptors, whereas D2Rs belong to inhibitory Ga_{i/olf} family of G-coupled protein receptors (Girault, J. A. (2012) Adv. Exp. Med. Biol. 970:407-429). To test this hypothesis, Applicant measured protein densities of D1Rs, D2Rs, and phosphorylation states of major downstream targets in striatum. P2X4R KO mice-exhibited significant increases in D1R protein density by 159% [p<0.05; FIG. 1A and FIG. 1B(iii)] and in D2R protein density by 42% [p<0.05; FIG. 1A and FIG. 1B (iv)] in the ventral striatum as compared to WT mice. There were no changes in either of protein densities for DA receptors in the dorsal striatum of P2X4R KO mice.

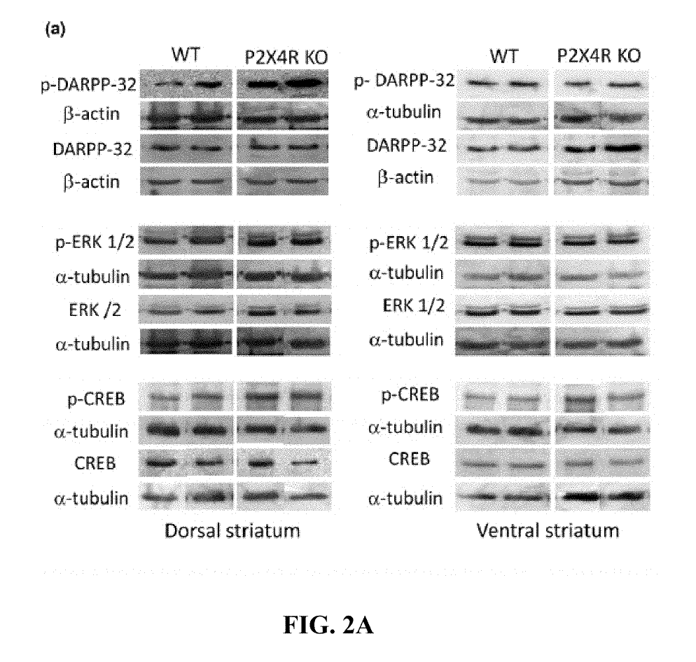

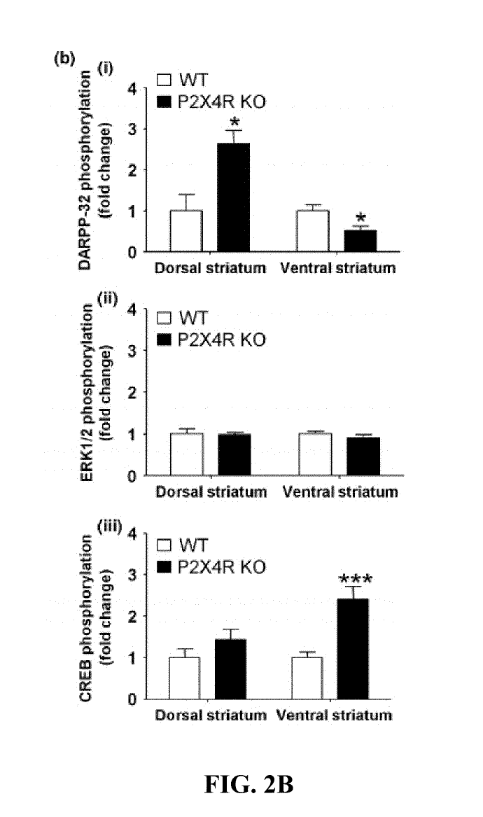

To investigate downstream pathways regulated by DA receptors, Applicant measured total and phosphorylated form of DARPP-32, ERK 1/2, and CREB in the dorsal and ventral striatum using western immunoblotting. P2X4R KO mice exhibited a significant increase in DARPP-32 phosphorylation (by 164%, p<0.05) without any changes in total DARPP-32 protein density in the dorsal striatum as compared to WT counterparts [FIG. 2A and FIG. 2B (i)]. In the ventral striatum, P2X4R KO mice exhibited a significant decrease in DARPP-32 phosphorylation (by 48%, p<0.05) without any changes in protein density of total DARPP-32 [FIG. 2A and FIG. 2B (i)]. P2X4R KO mice did not exhibit any significant changes in total ERK 1/2 protein density or ERK 1/2 phosphorylation in the dorsal and ventral striatum compared to WT mice [FIG. 2A and FIG. 2B (ii)]. There were no significant changes in protein density of total CREB or CREB phosphorylation in the dorsal striatum of P2X4R KO mice [FIG. 2A and FIG. 2B (iii)]. On the other hand, P2X4R KO mice did exhibit a significant decrease in total CREB density (by 36%, p<0.01) and a corresponding increase in CREB phosphorylation (by 141%, p<0.001) in the ventral striatum compared to WT mice [FIG. 2A and FIG. 2B (iii)].

### IVM Significantly Affected DARPP-32 and ERK 1/2 Phosphorylation, but not CREB Phosphorylation in the Dorsal Striatum

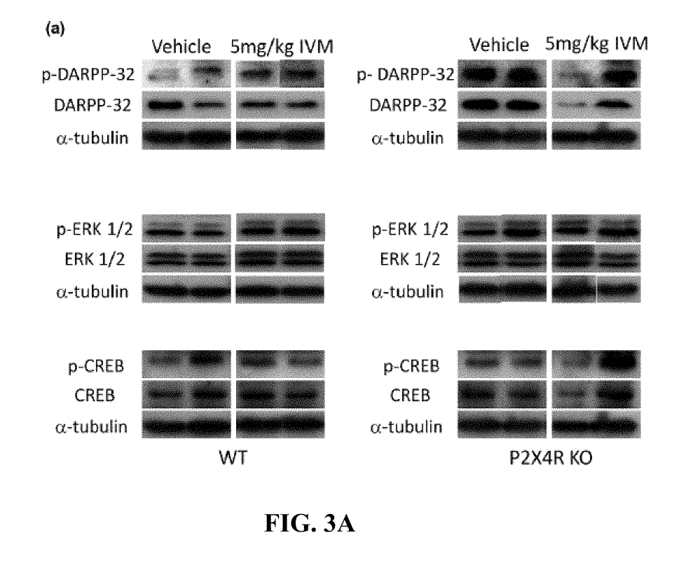

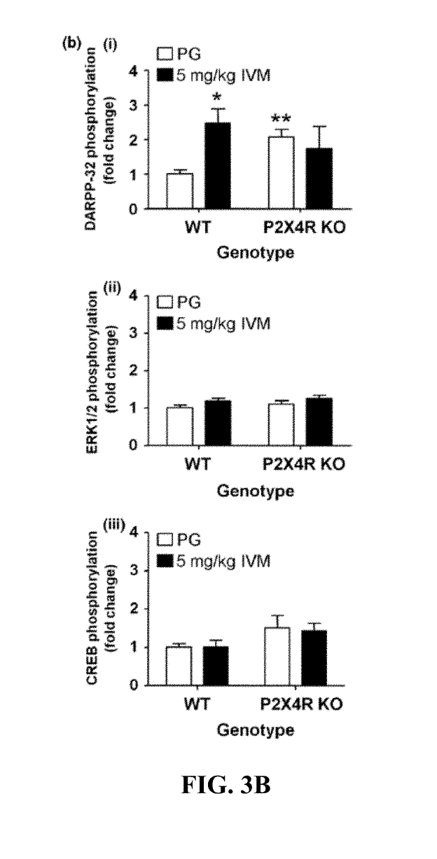

IVM was used to gain pharmacological insights into the role of P2X4Rs in dopaminergic signaling in the dorsal striatum. There was no significant effect of IVM treatment or genotype on total DARPP-32 levels or phosphorylation, but the drug treatment×genotype interaction was significant for both the total DARPP-32 levels [F(1,18)=5.61, p<0.05] and DARPP-32 phosphorylation [F (1,18)=5.48, p<0.05]. Bonferroni post hoc test confirmed significant increase in DARPP-32 phosphorylation upon IVM treatment in WT (t=2.834, p<0.05), but not in P2X4R KO mice (t=0.5829, p>0.05) [FIG. 3A and FIG. 3B(i)]. There was no significant effect of IVM treatment or genotype on total ERK 1/2 levels, but the drug treatment×genotype interaction was significant [F (1,18)=10.00, p<0.01]. There was a significant effect of IVM treatment [F (1,18)=4.62, p<0.05] but not genotype or drug treatment×genotype interaction for ERK 1/2 phosphorylation [FIG. 3A and FIG. 3B (ii)]. There was no significant effect of IVM treatment, genotype, or drug treatment×genotype interaction for total CREB levels. There was no significant effect of IVM treatment or drug treatment×genotype interaction, but the effect treatment×genotype interaction was significant for both the total DARPP-32 levels [F (1,18)=5.61, p<0.05] and DARPP-32 phosphorylation [F (1,18)=5.48, p<0.05]. Bonferroni post hoc test confirmed significant increase in DARPP-32 phosphorylation upon IVM treatment in WT (t=2.834, p<0.05), but not in P2X4R KO mice (t=0.5829, p>0.05) [FIG. 3A and FIG. 3B(i)]. There was no significant effect of IVM treatment or genotype on total ERK 1/2 levels, but the drug treatment×genotype interaction was significant [F(1,18)=10.00, p<0.01]. There was a significant effect of IVM treatment [F(1,18)=4.62, p<0.05] but not genotype or drug treatment×genotype interaction for ERK 1/2 phosphorylation [FIG. 3A and FIG. 3B (ii)]. There was no significant effect of IVM treatment, genotype, or drug treatment×genotype interaction for total CREB levels. There was no significant effect of IVM treatment or drug treatment×genotype interaction, but the effect of genotype trended toward significance for CREB phosphorylation [F(1,16)=4.37, p=0.0529] [FIG. 3A and FIG. 3B (iii)].

### IVM Significantly Affected DARPP-32 Phosphorylation, but not ERK 1/2 or CREB Phosphorylation in the Ventral Striatum

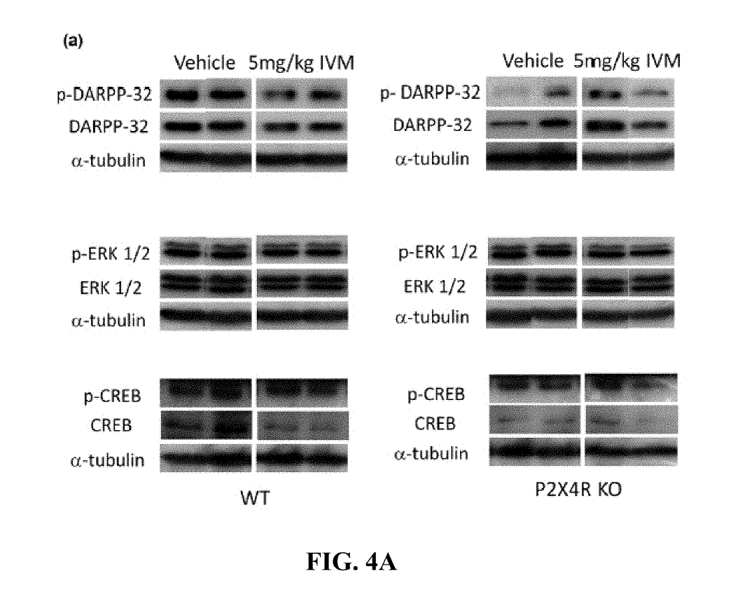

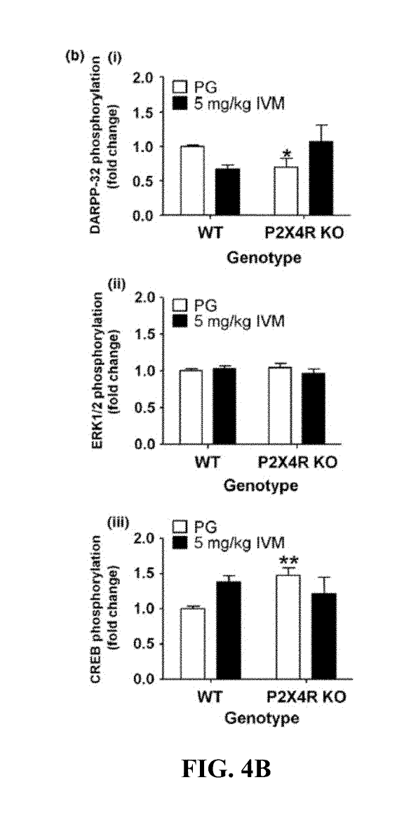

Similar to dorsal striatum, Applicant investigated the effect of IVM on phosphorylation of the same signaling molecules in the ventral striatum. There was no significant effect of IVM treatment or genotype on total DARPP-32 levels or DARPP-32 phosphorylation, but the drug treatment×genotype interaction was significant for both total DARPP-32 levels [F (1,16)=6.40, p<0.05] and DARPP-32 phosphorylation [F(1,16)=6.20, p<0.05] [FIG. 4A and FIG. 4B (i)]. There was no significant effect of IVM treatment, genotype, or drug treatment×genotype interaction for total ERK 1/2 levels or ERK 1/2 phosphorylation [FIG. 4A and FIG. 4B(ii)]. There was no significant effect of IVM treatment, genotype, or drug treatment×genotype interaction for total CREB levels. Similarly, there was no significant effect of IVM treatment or genotype on CREB phosphorylation. However, the drug treatment×genotype interaction trended toward significance for CREB phosphorylation [F (1,15)=4.43, p=0.0527] [FIG. 4A and FIG. 4B (iii)].

### Pharmacological Inhibition of D1Rs and D2Rs Significantly Enhanced Prepulse Inhibition (PPI) of Acoustic Startle Reflex in P2X4R KO Mice

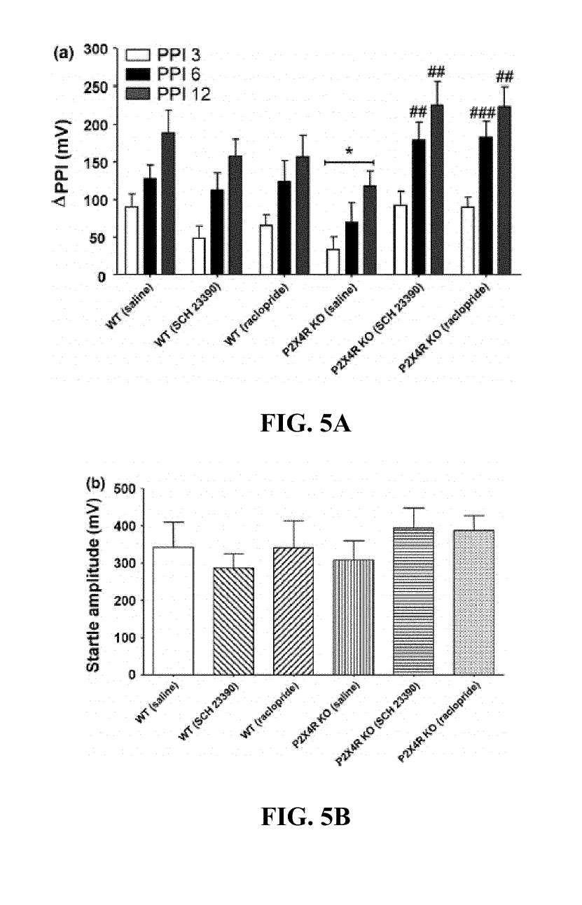

Applicant investigated the response of P2X4R KO mice to selective antagonists for D1Rs (SCH-23390; 1 mg/kg) and D2Rs (raclopride; 3 mg/kg) on PPI startle response, to link imbalances in DA homeostasis to PPI deficits in P2X4R KO mice. The doses tested were chosen based on previous studies that investigated PPI function in C57BL/6 mice (Ralph, R. J. et al. (2001) J. Neurosci. 21:305-313; Ralph-Williams, R. J. et al. (2003) Neuropsychopharmacology 28:108-118; Doherty, J. M. et al. (2008) Neuropsychopharmacology 33:2648-2656). Applicant found a significant effect of genotype on PPI function [F(1,26)=5.50, p<0.05] (FIG. 5A) which supports previous findings (Wyatt, L. R. et al. (2013) Neuropsychopharmacology 38:1993-2002). SCH-23390 significantly increased PPI in P2X4R KO mice [F(1,28)=9.33, p<0.01] with Bonferroni post hoc test identifying a significant increase at PPI 6 (t=3.294, p<0.01) and PPI 12 (t=3.240, p<0.01) (FIG. 5A). Similarly, raclopride significantly enhanced PPI function in P2X4R KO mice [F(1,28)=11.98, p<0.01] as compared to saline-treated P2X4R KO mice with Bonferroni post hoc test identifying a significant increase at PPI 6 (t=3.748, p<0.001) and PPI 12 (t=3.507, p<0.01) (FIG. 5A). There were no significant changes in PPI function in WT mice upon treatment with SCH-23390 or raclopride (FIG. 5A). Moreover, neither SCH-23390 nor raclopride induced any significant change in startle amplitude in P2X4R KO mice (FIG. 5B).

### Pharmacological or Genetic Manipulation of P2X4R Function Significantly Influenced L-DOPA-Induced Motor Behavior in the 6-OHDA Model of DA Depletion

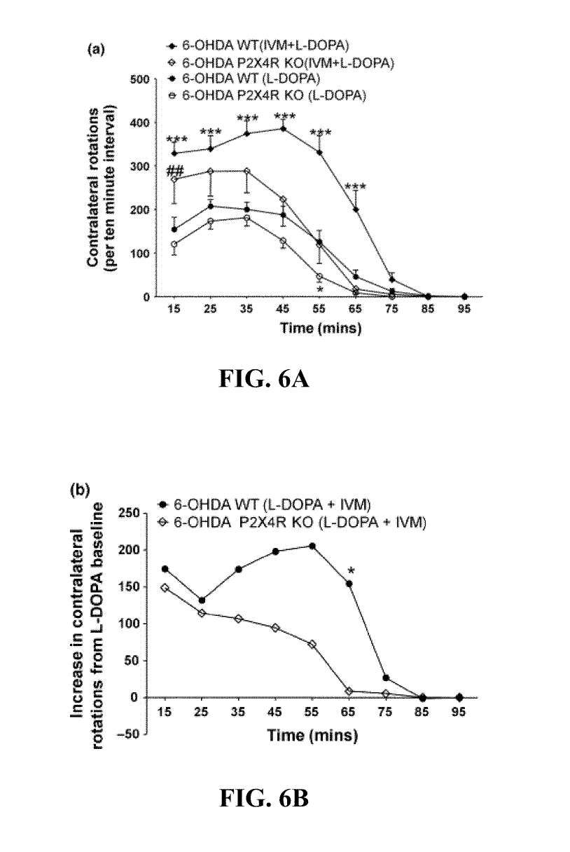

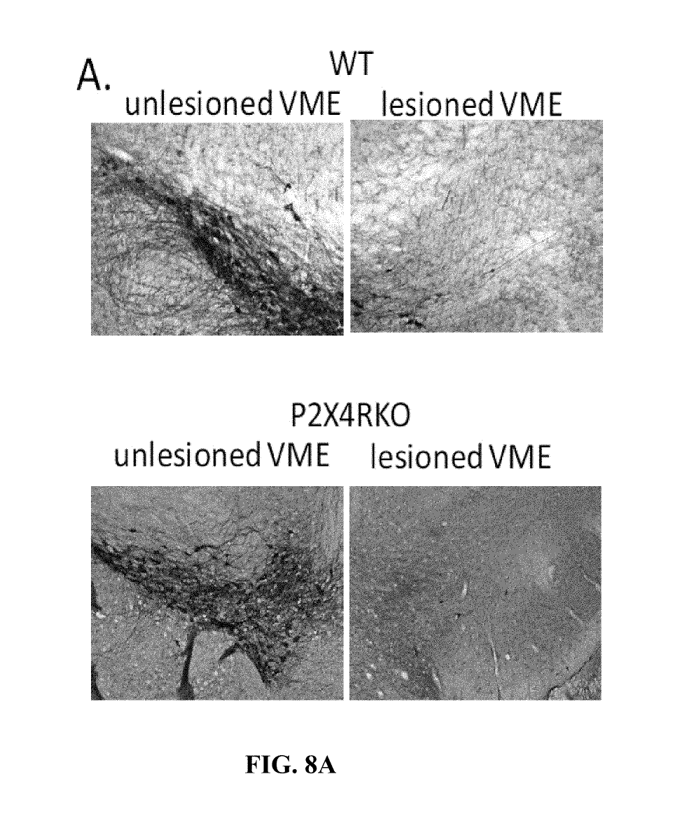

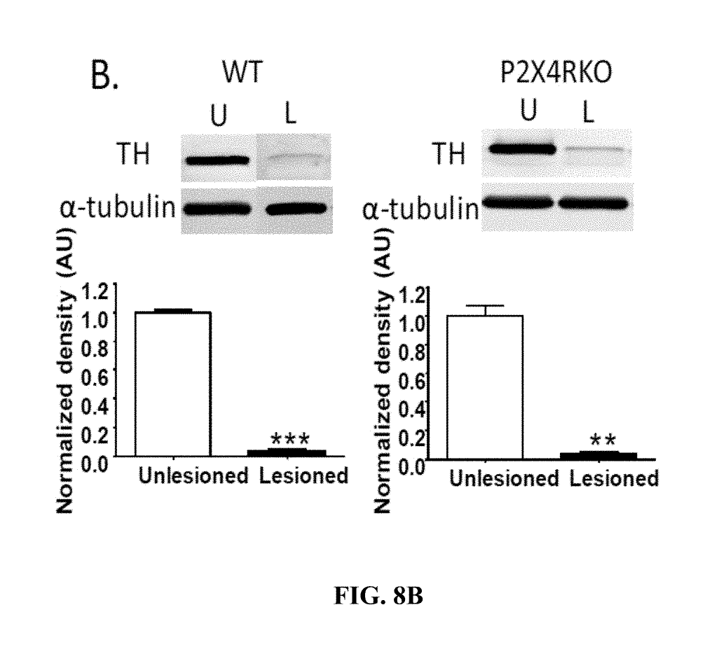

Using the 6-OHDA model of DA depletion, Applicant investigated the role of P2X4Rs in regulation of motor behavior (Schwarting, R. K. et al. (1996) Prog. Neurobiol. 50:275-331).× As presented, ablation of DA neurons in ventral mesencephelon in both genotypes (FIG. 8) resulted in reduced TH expression by 95.7% and 96.3% in the striatum of WT (p<0.001) and P2X4R KO mice (p<0.01), respectively (FIG. 8). In the presence of L-DOPA treatment (5 mg/kg), both genotypes exhibited contralateral rotations, as L-DOPA metabolizes into DA in the synapses, followed by activation of the supersensitive post-synaptic DA receptors on lesioned striatum (Ungerstedt, U. (1971) Acta Physiol. Scand. Suppl. 367:69-93) (FIG. 6A). In the presence of L-DOPA, Applicant found that P2X4R KO mice exhibited significantly fewer contralateral turns as compared to WT mice (FIG. 6A). There was a significant effect of genotype [F(1,20)=4.39, p<0.05] and time [F(8,160)=58.46, p<0.001] on L-DOPA-induced rotational behavior in P2X4R KO. There was no significant time×genotype interaction for L-DOPA-induced motor behavior. Bonferroni post hoc test confirmed significant reduction in L-DOPA-induced motor behavior in P2X4R KO mice during the 45-55 min interval (t=3.154, p<0.05) (FIG. 6A).

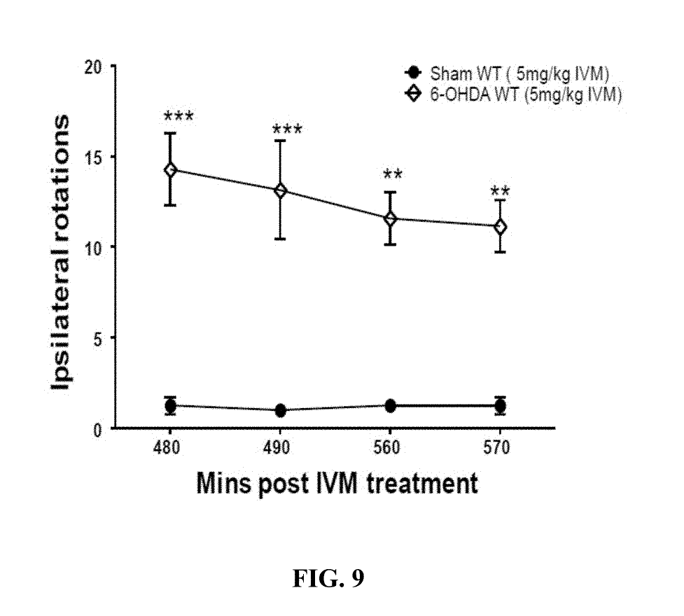

Applicant reasoned that if genetic disruption of P2X4Rs reduces L-DOPA response on motor behavior, then pharmacological potentiation of P2X4Rs should enhance motor behavior. In support of this hypothesis, Applicant found that IVM (5 mg/kg) significantly increased the number of L-DOPA-induced rotations in WT mice. There was a significant effect of time [F_{(8,208)}=95.32, p<0.001] and IVM treatment [F_{(1,26)}=33.80, p<0.001] on L-DOPA-induced motor behavior. There was also a significant time×drug treatment interaction [F_{(8,208)}=10.99, p<0.001] with Bonferroni post hoc test identifying a significant increase in contralateral rotations upon IVM treatment during 5-15 min (t=5.509, p<0.001), 15-25 min (t=4.161, p<0.001), 25-35 min (t=5.491, p<0.001), 35-45 min (t=6.254, p<0.001), 45-55 min (t=6.484, p<0.001), and 55-65 min (t=4.878, p<0.001) intervals (FIG. 6A). IVM alone induced an average of 12 rotations±2 rotations (SEM) for period of 2 hours post-drug treatment, although it was still statistically significant compared to sham controls [F_{(1,9)}=26.64, p<0.001] (FIG. 9).

There was a significant effect of time [F_{(8,112)}=36.93, p<0.001] and IVM treatment [F_{(1,14)}=4.80, p<0.05] on L-DOPA-induced motor behavior in P2X4R KO mice (FIG. 6A). There was also a significant time×drug treatment interaction [F_{(8,112)}=2.88, p<0.01] with Bonferroni post hoc test confirming a significant increase in L-DOPA induced motor behavior during the 5-15 min interval (t=3.481, p<0.01). Taking into consideration the lower L-DOPA baseline in P2X4R KO mice, Applicant analyzed the difference in the number of contralateral rotations between L-DOPA alone and IVM+L-DOPA in WT and P2X4R KO mice. There was a significant effect of time [F_{(8,160)}=11.33, p<0.001] and a non-significant trend of effect of genotype [F_{(1,20)}=3.17, p=0.0903] on contralateral rotations between L-DOPA and L-DOPA+IVM (FIG. 6B). There was a significant time×genotype interaction [F_{(8,160)}=2.07, p<0.05], suggesting that IVM's ability to increase L-DOPA-induced motor behavior differed between WT and P2X4R KO mice. Bonferroni post hoc test identified significant differences in L-DOPA-induced motor behavior upon IVM treatment between WT and P2X4R KO mice at 55-65 min (t=2.948, p<0.05) (FIG. 6B).

### Discussion

This example investigated the role of P2X4Rs in the dopaminergic system and its impact on DA-dependent behaviors. Impairments in DA neurotransmission were observed with respect to changes in protein densities of pre- and post-synaptic markers. There was increased TH protein density in the dorsal, but not the ventral, striatum of P2X4R KO mice. DAT was significantly increased in both parts of the striatum in P2X4R KO mice, which is indicative of higher pre-synaptic reuptake of DA. In addition to changes in pre-synaptic markers of DA activity, Applicant also identified significant changes in protein densities of DA receptors in the ventral, but not the dorsal, striatum of P2X4R KO mice. The increased DA receptors' density in the ventral striatum could be an adaptive response to the altered synaptic DA availability due to increased DAT expression in the same brain region of P2X4R KO mice. This interpretation is based on previous studies that have postulated a positive correlation between DAT and DA receptor density (Fauchey, V. et al. (2000) Eur. J. Neurosci. 12:19-26; Ghisi, V. et al. (2009) Cell. Signal. 21:87-94). The increased DAT and DA receptor density levels suggest that P2X4R KO mice may have alterations in DA neurotransmission. However, there were no significant changes in DA levels in both parts of the striatum of P2X4R KO mice despite significant alterations in TH density levels in the dorsal striatum of P2X4R KO mice. There are several other factors that control DA levels such as storage, release, reuptake, and catabolism (Eells, J. B. (2003) Curr. Med. Chem. 10:857-870). Overall, the findings suggest that p2rx4 deficiency affects DA synthesis and transport that could impact normal DA neuron function.

In addition to DA receptors, Applicant identified dysregulation of signaling molecules (i.e., DARPP-32 and CREB) that can be regulated through DA receptors in different striatal regions of P2X4R KO mice. D1R stimulation on striatonigral MSNs phosphorylates DARPP-32 at Thr34 via protein kinase A that inhibits protein phosphatase-1 activity and allows phosphorylation of ERK 1/2 and CREB. D2R stimulation produces the opposite effects in the striatopallidal MSNs (Girault, J. A. (2012) Adv. Exp. Med. Biol. 970:407-429; Bertran-Gonzalez, J. et al. (2008) J. Neurosci. 28:5671-5685). Despite the lack of significant changes in D1Rs protein density, P2X4R KO mice exhibited a significant increase in DARPP-32 phosphorylation in the dorsal striatum, which is typically indicative of up-regulated D1R-mediated signaling function. On the other hand, Applicant saw a significant decrease in DARPP-32 phosphorylation in the ventral striatum of P2X4R KO mice that correlates well with the increased D2R protein density in the same brain region. But, significant increases in D1R density and CREB phosphorylation in the same brain region of P2X4R KO mice that did not corroborate with the decreased DARPP-32 phosphorylation were observed. One possible explanation for these neurochemical differences identified in the P2X4R KO mice is that there are multiple interactions or involvement of various neurotransmitter systems besides DA in regulating DARPP-32 phosphorylation such as glutamate, GABA, and serotonin (Svenningsson, P. et al. (2004) Annu. Rev. Pharmacol. Toxicol. 44:269-296). Thus, the alterations in signaling molecules including DARPP-32 and CREB in P2X4R KO mice suggest a complex compensatory change other than that of DA receptors. Taken together; the increased density of pre- and post-synaptic markers suggests dysregulation of DA system in P2X4R KO mice which may partially underlie the behavioral deficits previously reported in P2X4R KO mice.

In addition to the genetic approach, Applicant used a pharmacological approach to explore a role for P2X4Rs in regulating DA receptor-associated signaling pathways. As there are limited specific antagonists to test P2X4R-related signaling in vivo, Applicant used the P2X4R allosteric modulator, IVM, to investigate a link between P2X4R function and DA receptor-associated signaling molecules in the dorsal and ventral striatum. As presented above, there was a significant increase in DARPP-32 phosphorylation upon IVM treatment in the WT, but not in P2X4R KO mice, suggesting a role for P2X4Rs in regulating DARPP-32 phosphorylation in the dorsal striatum. The changes in DARPP-32 phosphorylation upon P2X4R potentiation by IVM did not fully agree with the result from genetic deletion of P2X4Rs in the dorsal striatum. This difference in finding may be linked to neurodevelopmental changes in P2X4R KO mice. This hypothesis is supported by several lines of evidence: First, IVM did not increase DARPP-32 phosphorylation in P2X4R KO mice; second, P2X4Rs have been reported to be expressed from post-natal day 1 (Cheung, K. K. et al. (2005) Neuroscience 133:937-945); third, the P2X4R KO mice exhibit communication deficits during their pre-adult period (Wyatt, L. R. et al. (2013) Neuropsychopharmacology 38:1993-2002). As phospho-Thr34-DARPP-32 can indirectly increase ERK 1/2 phosphorylation in the striatum (Girault, J. A. (2012) Adv. Exp. Med. Biol. 970:407-429), it was not surprising to see a significant effect of IVM treatment on ERK 1/2 phosphorylation. However, increase in ERK 1/2 phosphorylation was seen in both the genotypes, indicating that IVM's ability to modulate ERK1/2 phosphorylation is independent of P2X4R function. Interestingly, there was a significant interaction between IVM treatment and genotype for total ERK 1/2 levels, indicating that effect of IVM on total ERK 1/2 expression was dependent upon P2X4R function. Hence, IVM might be increasing ERK 1/2 phosphorylation in the WT mice by regulating total expression of the protein upon P2X4R potentiation. Although previous investigations have used IVM as a pharmacological tool for studying P2X4R function in vitro and in vivo (Sim, J. A. et al. (2006) J. Neurosci. 26:9006-9009; Asatryan, L. et al. (2010) J. Pharmacol. Exp. Ther. 334:720-728; Bortolato, M. et al. (2013) Int. J. Neuropsychopharmacol. 16:1059-1070; Popova, M. et al. (2013) Purinergic Signal. 9:621-632), IVM does have other protein targets in the CNS including GABA_{A} receptors (Dawson, G. R. et al. (2000) J. Pharmacol. Exp. Ther. 295:1051-1060), nicotinic acetylcholine receptors (Krause, R. M. et al. (1998) Mol. Pharmacol. 53:283-294), and glycine receptors (Shan, Q. et al. (2001) J. Biol. Chem. 276:12556-12564). Similar to the dorsal striatum, IVM modulated DARPP-32 phosphorylation via P2X4R potentiation in the ventral striatum. Moreover, IVM had a tendency to differentially modulate CREB phosphorylation via P2X4R activity in the same brain region.

The significant increases in DA receptor protein density in the ventral striatum reported herein may underlie the PPI deficits in P2X4R KO mice. PPI measures reduction in startle reflex that occurs when the eliciting acoustic burst is immediately preceded by a weak stimulus and is highly reliable index for measuring sensorimotor gating (Ison, J. R. et al. (1983) Psychol. Bull. 94:3-17). Multiple findings have reported a critical role for DA receptors in regulation of PPI function, of which D2Rs have received considerable attention on basis of findings from pharmacological studies in rats and patient population (Swerdlow, N. R. et al. (1991) J. Pharmacol. Exp. Ther. 256:530-536; Abduljawad, K. A. et al. (1998) J. Psychopharmacol. 12:239-245; Kumari, V. et al. (1998) Behay. Pharmacol. 9:567-576; Volter, C. et al. (2012) Int. J. Neuropsychopharmacol. 15; 1427-1440). However, gene knockout and pharmacological studies in mice have implicated both D1Rs and D2Rs (Ralph-Williams, R. J. et al. (2002) J. Neurosci. 22:9604-9611; Ralph-Williams, R. J. et al. (2003) Neuropsychopharmacology 28:108-118; Doherty, J. M. et al. (2008) Neuropsychopharmacology 33:2648-2656). In the context of findings from the literature, Applicant used both D1R (SCH-23390) and D2R (raclopride) antagonists to identify potential contribution of DA receptors to PPI functioning in P2X4R KO mice. Applicant discovered that the PPI deficits in P2X4R KO mice were significantly ameliorated by SCH-23390 and raclopride, indicating D1Rs and D2Rs as important modulators of PPI function in mice. The increased density of DA receptors in the ventral striatum, integral to corticolimbic-striato-pallidal circuitry of PPI (Swerdlow, N. R. et al. (2008) Psychopharmacology 199:331-388), of P2X4R KO mice may contribute to PPI dysfunction and that blocking these receptors can reverse the deficit. The pharmacological studies provide insights into the functional consequences of altered DA receptor protein densities on behaviors such as sensorimotor gating in the P2X4R KO mice. Moreover, Applicant discovered IVM-mediated PPI disruption in WT C57BL/6J mice and its attenuated response in P2X4R KO mice, which further supports a role for P2X4Rs in sensorimotor gating (Bortolato, M. et al. (2013) Int. J. Neuropsychopharmacol. 16:1059-1070). Overall, these studies identify potential interactions between P2X4R function and DA neurotransmission in regulating sensorimotor gating.

The neurochemical and behavioral alterations in P2X4R KO mice could be relevant to multiple psychiatric disorders such as schizophrenia, attentional deficit hyperactivity disorder, Obsessive-Compulsive disorder, and bipolar depression. For example, post mortem studies have reported an increase in D2R and DAT expression in psychotic and non-psychotic disorders (Pearlson, G. D. et al. (1995) Arch. Gen. Psychiatry 52:471-477; Krause, K. H. et al. (2000) Neurosci. Lett. 285:107-110; Brunswick, D. J. et al. (2003) Am. J. Psychiatry 160:1836-1841; Perez, V. et al. (2003) Prog. Neuropsychopharmacol. Biol. Psychiatry 27:767-770). In addition, increased TH expression and pre-synaptic DA synthesis has been reported in neuroleptic naïve psychotic patients (Hietala, J. et al. (1995) Lancet 346:1130-1131; Hietala, J. et al. (1999) Schizophr. Res. 35:41-50). Applicant observed increased density in D2Rs in the ventral striatum, DAT in both striatal regions and TH in the dorsal striatum in P2X4R KO mice. Applicant identified increased D2Rs in the striatum. In addition, P2X4R KO mice exhibited PPI deficits. Interestingly, the up-regulation of D2Rs, altered sensitivity to DA receptor acting drugs, and PPI deficits in P2X4R KO mice correlate with findings from mouse models linked to psychiatric disorders (Ralph, R. J. et al. (2001) J. Neurosci. 21:305-313; Kinkead, B. et al. (2005) J. Pharmacol. Exp. Ther. 315:256-264; Wolinsky, T. D. et al. (2007) Genes Brain Behay. 6:628-639; Lipina, T. V. et al. (2010) Genes Brain Behay. 9:777-789).

Applicant's findings also suggests that P2X4Rs are involved in other DA-dependent functions of the basal ganglia including motor behavior. To test this, Applicant used the 6-OHDA animal model in combination with IVM and P2X4R KO mice, which is a well-established model for elucidating DA interactions with other neurotransmitter systems in motor activity (Fox, S. H. et al. (2000) Eur. J. Pharmacol. 398:59-64; Xiao, D. et al. (2011) Brain Res. 1367:310-318). Also, this model is used for understanding the pathogenesis of movement disorders including Parkinson's disease and screening of novel therapeutics (Schwarting, R. K. et al. (1996) Prog. Neurobiol. 50:275-331; Deumens, R. et al. (2002) Exp. Neurol. 175:303-317). Applicant found that the L-DOPA-induced motor behavior was significantly decreased in 6-OHDA lesioned P2X4R KO mice, indicating that disruption of P2X4R function significantly affected L-DOPA-induced behavioral response. This attenuated response in P2X4R KO mice may be due to alterations in DA system in striatonigral circuitry of the basal ganglia. Alternatively, L-DOPA attenuated response could be linked to its faster clearance or metabolism in P2X4R KO mice. This hypothesis will be explored in future studies. Conversely, Applicant demonstrated that pharmacological modulation of P2X4R activity by IVM significantly enhanced L-DOPA-induced rotational behavior. A plausible mechanism underlying L-DOPA+IVM response is a synergy between P2X4Rs and D1Rs on MSNs in disinhibition of neurons projecting from the substantia nigra pars reticulate (SNR) to the thalamus, superior colliculus, and pendenculopontine nucleus and, thereby, producing contralateral rotations.

Notably, increased expression of P2X4Rs has been reported in the MSNs of SNR of 6-OHDA-treated rats (Amadio, S. et al. (2007) Purinergic Signal. 3:389-398) and so, these compensatory changes may partially explain the augmented L-DOPA-dependent motor response in presence of IVM. IVM did not influence motor behavior independently, suggesting that activation of P2X4Rs alone is not sufficient enough to cause disinhibition of SNR to induce such a response. Unlike D1Rs that are present exclusively on post-synaptic MSNs, P2X4Rs are present both on pre-synaptic DA neurons and post-synaptic MSNs. The simultaneous activation of P2X4Rs at the presynapses and postsynapses would counteract each other, thus preventing the mice from turning to either side. The lack of effect with IVM alone supports the notion that IVM has a modulatory effect on L-DOPA's motor behavior. In addition to increased behavioral response in WT mice, IVM also enhanced L-DOPA's motor behavior in P2X4R KO mice. However, while comparing the absolute increase in LDOPA's motor behavior in presence of IVM, Applicant saw a significant interaction between time and genotype, suggesting an altered effect of IVM between WT and P2X4R KO mice. This finding supports Applicant's previous finding that IVM mediates its behavioral effects partially via action on P2X4Rs (Bortolato, M. et al. (2013) Int. J. Neuropsychopharmacol. 16:1059-1070; Wyatt, L. R. et al. (2014) Neurochem. Res. 39:1127-1139). Nevertheless, the increase in L-DOPA response in P2X4R KO mice suggests that IVM may be modulating L-DOPA response through a complex network of receptor systems. Overall, the findings suggest that P2X4Rs have a synergistic role in DA modulation of motor control and can alter behavioral responses to dopaminergic drugs. As such, P2X4R allosteric modulators may represent potential adjuvant pharmacotherapies for Parkinson's disease.

In conclusion, this investigation supports the hypothesis that there are signaling interactions between P2X4Rs and DA neurotransmission in regulation of multiple CNS functions in the basal ganglia. Finally, although at nascent stage, these findings implicate P2X4Rs in neurobiological mechanisms of multiple neurological disorders. There is growing interest in P2XRs as novel drug targets for therapeutic development in psychiatry disorders (Ortiz, R. et al. (2015) Prog. Neuropsychopharmacol. Biol. Psychiatry 57:117-131). In support of this hypothesis, others report that the gene for P2X4Rs is located in chromosome 12q24 (Gu, B. J. et al. (2010) Mol. Biol. Rep. 37:3369-3376) which contains several loci that can alter susceptibility to schizophrenia, bipolar disorder, and attention deficit hyperactivity (Dawson, E. et al. (1995) Am. J. Med. Genet. 60:94-102; Bailer, U. et al. (2000) Neuropsychobiology 42:175-182; Jones, I. et al. (2002) Mol. Psychiatry 7:424-427). Non-synonymous single nucleotide polymorphisms in p2rx4 gene have been linked to high pulse pressure and age-related macular degeneration (Caseley, E. A. et al. (2014) Int. J. Mol. Sci. 15:13344-13371).

### Example 2: Reduction of P2X4 Receptors to Reduce Ethanol Intake

### Introduction

In Example 1 it was shown discussed that P2X4R knockout (KO) mice (i.e., p2rx4 deleted) deficits in ethanol consumption compared to wildtype (WT) controls (Wyatt, L. R. et al. (2014) Neurochem. Res. 39:1127-1139). In the present example, the length of time of the ethanol investigation was increased relative to Wyatt, L. R. et al. (2014) Neurochem. Res. 39:1127-1139, to gain insights regarding the transient nature of the increased drinking reported by Wyatt and colleagues. This was accomplished by testing male P2X4R KO mice and WT littermates for changes in ethanol intake and preference for 5 weeks using a 24-h access two-bottle choice paradigm. Second, given that the increase in ethanol intake previously measured in male P2X4R KO mice could partially reflect compensatory developmental changes, a lentiviral-mediated shRNA knockdown strategy (LV-shRNA) was utilized to knockdown P2X4R expression in the NAc core, and then changes in ethanol intake and preference were measured.

### Materials and Methods

### Animals

Naïve 2-3 month old male WT and P2X4R KO mice from the breeding colony at the University of Southern California were used. The generation of P2X4R KO mice and the breeding scheme are described in Sim, J. A. et al. (2006) J. Neurosci. 26:9006-9009 and Wyatt, L. R. et al. (2013) Neuropsychopharmacology 38:1993-2002. For the LV-shRNA experiments, 2-3 month old male C57BL/6 mice were obtained from Jackson Laboratories (Bar Harbor, Me.). Mice were group-housed (i.e., five per cage) in the vivarium maintained at 22° C. and under a 12-h/12-h light/dark cycle with ad libitum access to food and water. All procedures were carried out in compliance with the guidelines of the National Institutes of Health and approved by the Institutional Animal Care and Use Committee of the University of Southern California.

### Drugs

The ethanol solution was prepared as a 10% v/v ethanol solution in tap water from 190 proof USP grade ethanol solution (Koptec; King of Prussia, Pa.).

Short Hairpin RNA (shRNA) Constructs and LV Production

cDNA encoding two shRNA sequences targeting different regions of P2X4 mRNA (S1 and S2) were subcloned into a Clontech biscistronic pLVX-shRNA2 vector, in which the shRNA expression was driven by human U6 promoter, located just upstream of the MCS (Mountain View, Calif.). The vector also expressed ZsGreen1 reporter, a human-codon optimized variant of the coral reef green fluorescent protein (GFP), under CMV promoter control. The shRNA sequences were 5′-CCACAAATACTCAGGGTTG-3′ and 5′-CTCAGATGGGCTTCAGATA-3′. Applicant has observed that the simultaneous use of both of the sequences resulted in a higher extent of inhibition of P2X4R expression. LV was produced by mixing both shRNA constructs with psPAX2 and pMD2. G packaging vectors, obtained from Addgene (Cambridge, Mass.) and from transfected HEK 293T cells. Virus-containing supernatant was collected and concentrated, and the resultant viral titers were determined via the ELISA method. Concentrator and titration kits were obtained from Clontech Laboratories (Mountain View, Calif.).

### Stereotaxic Surgery and Microinjection Procedure

Mice were anesthetized with a ketamine/xylazine cocktail and placed in a mouse stereotaxic frame (David Kopf Instruments; Tujunga, Calif.). A small incision was made to the skin to expose the skull. The bregma and lambda were measured to ensure an even plane, and a small area of dura was removed in the area for microinjection. A 10-μL syringe (Hamilton; Reno, Nev.) was used to deliver 1 μL of LV (4.1×10⁷ to 5.3×10⁹ IU/mL) to each NAc (bregma coordinates: anterior-posterior 1.2 mm; mediolateral 1.0 mm; dorsoventral 4.5 mm) at a rate of 0.1 μL/min. After infusion, the syringe was left in place for a further 5 min. Mice were allowed to recover from surgery in cages placed on heating pads for 2 days, after which they were transported to the vivarium. Mice were subsequently single-housed in the vivarium with ad libitum access to food and water during their resting period for 1 week prior to the start of the ethanol drinking experiments.

Anatomical Verification of LV Microinfusion into the NAc Core by Fluorescence Microscopy

Following the stereotaxic surgery and microinjection of LV, transcardial perfusion was performed on mice using 0.9% NaCl followed by 4% phosphate-buffered paraformaldehyde. Brains were post-fixed in 4% phosphate-buffered paraformaldehyde overnight, followed by storage in 20% sucrose for 48 h, and then frozen in 4-methylbutane on dry ice. Striatal sections were cut coronally at 25-μm thicknesses in a cryostat, and later stored in a cryoprotective solution containing 30% sucrose in phosphate-buffered saline at 4° C. until further use. The striatal sections were then examined for GFP immunofluorescence under a fluorescent microscope (Olympus BX61 microscope, Shinjuku; Tokyo, Japan).

### Verification of LV-shRNA-Mediated Knockdown of P2X4Rs In Vitro and In Vivo Using Western Immunoblotting

BV-2 transduction: Knockdown of P2X4Rs by LV-shRNA strategy in vitro was verified through transduction of mouse microglial BV-2 cells, which have a high endogenous P2X4R expression. BV-2 cells were cultured in 6-well plates in DMEM/F12 medium supplemented with penicillin/streptomycin and fetal bovine serum, until they reached approximately 80% confluence. Cells were transduced with 106 infectious units/mL of shRNA-based LV. Confirmation of virus expression was visualized by GFP fluorescence after 48 h.

Microinjection into NAc core: P2X4R knockdown in mouse NAc core by LV-shRNA methodology was verified at 14 and 42 days post-infusion of LV-shRNA. Mice were stereotactically injected with the LV-alone and LV-shRNA-p2rx4, respectively. A separate cohort of mice that had never undergone surgery (to be described as “naïve mice”) was used as a positive control for this study. After surgery, mice were allowed to rest for a period of 1 week. At the end of the recovery period, the mice remained in their cages for a period of 14 and 42 days, during which they had ad libitum access to food and water. Applicant chose the 14-day time point because the mice are exposed to ethanol 2 weeks post-surgery, and Applicant chose the 42-day time point because the final week during which they receive ethanol is 6 weeks post-surgery. At their respective time points, mice were euthanized using CO₂ asphyxiation, and the striatum was dissected out as per landmarks described in the mouse brain atlas (Franklin, B. J. and Paxinos, G. (2007) The Mouse Brain in Stereotaxic Coordinates. Elsevier Academic Press, Amsterdam).

BV-cell lysate or striatal tissue homogenate preparation: Cells or striatal tissues were treated with lysis buffer containing 50-mM tris-HCl at pH 7.4, 150-mM NaCl, 0.5% sodium deoxycholate, 1% Triton-X-100, 0.1% SDS, and 1% proteinase inhibitor cocktail (Millipore; Temecula, Calif.). BV-2 cell lysates were spun at 13,000 rpm for 10 min at 4° C., and the protein-containing supernatant was collected. Protein content for BV-2 cells and striatum was measured by using BCA protein assay (Thermo Scientific; Rockford, Ill.).

Immunoblotting procedure: Striatal homogenates or cell lysates (respectively, of 50 μg and 10 μg per lane) ran on 10% SDS PAGE gels and were transferred onto polyvinylidine fluoride membranes using a semi-dry transfer method (Trans turbo blot, BioRad; Hercules, Calif.). Non-specific binding was blocked using 5% nonfat dried milk (BioRad; Hercules, Calif.), followed by incubation with rabbit anti-P2X4 receptor antibody (Alomone Labs; Jerusalem, Israel) overnight at 4° C. Membranes were then incubated with goat anti-rabbit secondary antibody for 1 h at room temperature. Between the incubation steps for antibodies and blocking, membranes were washed three times, 5 min each time, with TBS containing 0.05% Tween-20. After secondary antibody incubation, membranes were incubated with ECL substrate, (BioRad; Hercules, Calif.), and bands were visualized using chemiluminescent method (ChemiDoc sys-tem, BioRad; Hercules, Calif.).

### 24-h Access Two-Bottle Choice Paradigm

The 24-h access two-bottle choice paradigm is a model that mimics social drinking and is used to investigate differences in ethanol intake in genetically modified animals or upon pharmacological treatment (Belknap, J. K. et al. (1993) Psychopharmacology (Berl) 112:503-510; Middaugh, L. D. et al. (1999) Alcohol 17:185-194; Yoneyama, N. et al. (2008) Alcohol 42:149-160). The 24-h access two bottle choice procedure used was that of Asatryan, L. et al. (2014) The International Journal of Neuropsychopharmacology 17:907-916; Wyatt, L. R. et al. (2014) Neurochemical Research 39:1127-1139; and Yardley, M. M. et al. (2012) Neuropharmacology 63:190-201. Single-housed WT/P2X4R KO or naïve/LV-alone/LV-shRNA-p2rx4 infused mice had 24-h access to two inverted graduated tubes (25 mL) with metal sippers positioned on stainless steel cage tops. Food was evenly distributed on the cage tops to avoid association with either of the tubes. The mice had access to tubes containing water only for the first week post-acclimation. In the second week, one of the tubes contained water and the other contained 10% ethanol solution (10E). 10E and water intakes were recorded by measuring the lower meniscus. It was ensured that positions of the tubes were switched every alternate day to avoid side preferences. Mice were given fresh solutions of 10E and water once a week. Body weights were measured and used to calculate the g/kg/24-h ethanol intake. Percent ethanol preference was determined by multiplying the ratio of volume of 10E intake (mL) over total fluid intake (10E and water) by 100.

For the LV-shRNA-p2rx4 drinking experiment, mice underwent a baseline drinking session post-acclimation during which their voluntary consumption of ethanol, water, total fluid intake, and ethanol preference was measured. Upon stable baseline drinking, mice were randomly assigned to one of the three treatment options: 1) LV-shRNA-p2rx4, LV-alone, or naïve mice (i.e., mice that had never undergone the surgery). ANOVA was used to ensure that the ethanol intake, preference, water, or total fluid intake did not significantly differ between the three treatment groups.

### Statistical Analyses

Repeated-measures two-way analysis of variance (ANOVA) (genotype×week) was used to investigate differences in 10E intake, 10E preference, water intake, and total fluid intake between WT and P2X4R KO mice, followed by a Bonferroni post hoc test for multiple comparisons. For the LV-shRNA drinking studies, separate two-way ANOVAs followed by Bonferroni post hoc comparisons were conducted between LV-alone and LV-shRNA-p2rx4 groups to analyze the effect of LV-shRNA on drinking behavior, as well as between naïve mice and the LV-alone group to determine whether injection of LV alone had any impact on drinking behavior. One-way ANOVA with Tukey's post hoc test was also used to compare the efficiency of LV-shRNA-p2rx4 transfusion and transfection on P2X4R knockdown in mouse striatum and BV-2 cells. Significance was set at p<0.05. All data were analyzed using Graph Pad software (Prism; San Diego, Calif.).

### Results

### P2X4R KO Mice Exhibited Increased Voluntary Ethanol Consumption in a 24-h Access Two Bottle Choice Paradigm

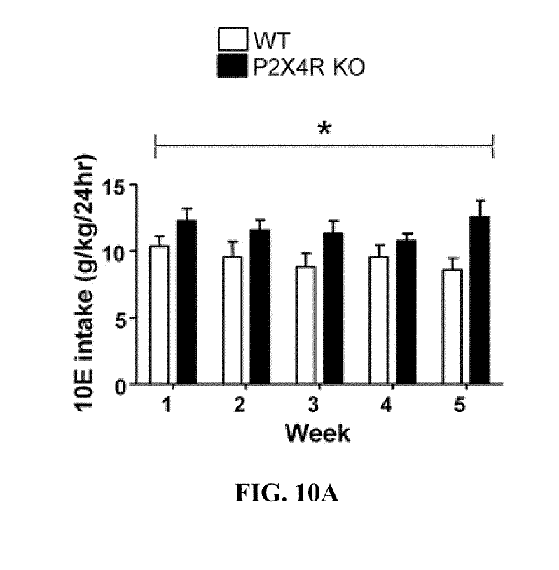

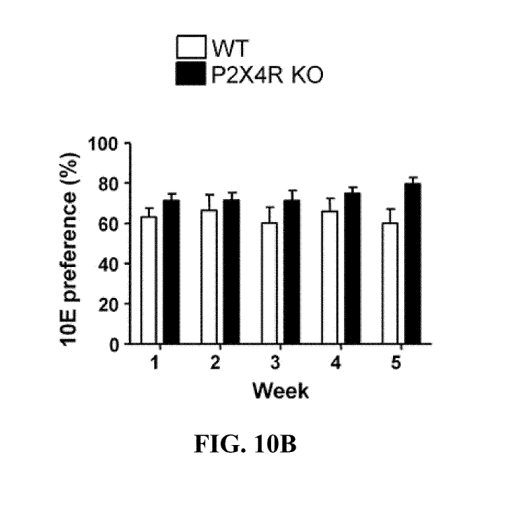

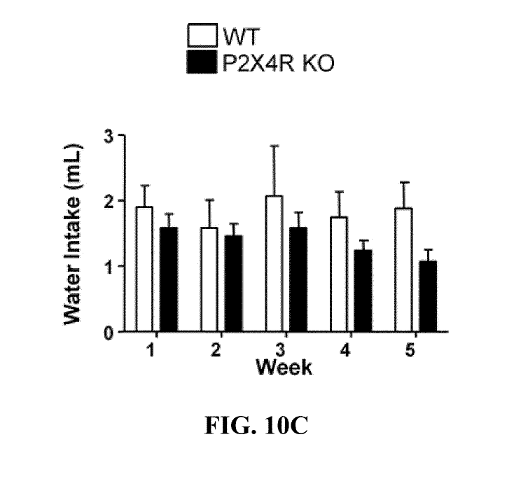

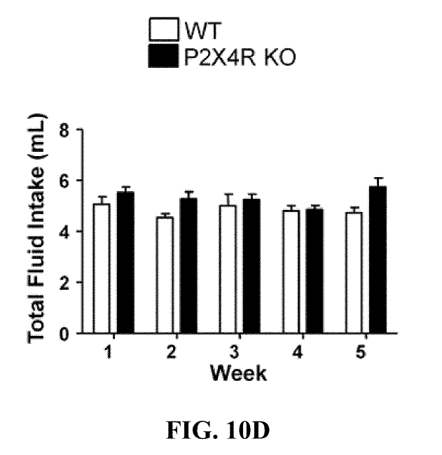

The effects of global knockout of the p2rx4 gene on ethanol intake using a 24-h access two-bottle choice paradigm (10E versus water) was monitored. As illustrated in FIG. 10A, there was a significant effect of genotype (F_{(1,16)}=4.88, p<0.05), but not week or genotype×week interaction for 10E intake. There was no significant effect of genotype, week, or genotype×week interaction for 10E preference or water intake between WT and P2X4R KO mice (FIGS. 10B and 10C). There was a non-significant trend toward effect of genotype (F_{(1,16)}=3.28, p=0.0891), but not week or genotype×week interaction for total fluid intake. (FIG. 10D). Considering that there were changes in 10E intake, but not preference, Applicant evaluated the effect of genotype and week on body weight. There was a significant effect of genotype (F_{(1,16)}=4.68, p<0.05) because the P2X4R KO mice weighed significantly more than their WT counterparts. There was also a significant effect of week (F_{(4,64)}=59.08, p<0.001) on body weight. There was no significant interaction between the two factors on body weight between WT and P2X4R KO mice.

Transfection of BV-2 Cells or Transfusion in Mouse Striatum with LV-shRNA-p2rx4 Reduced P2X4R Expression

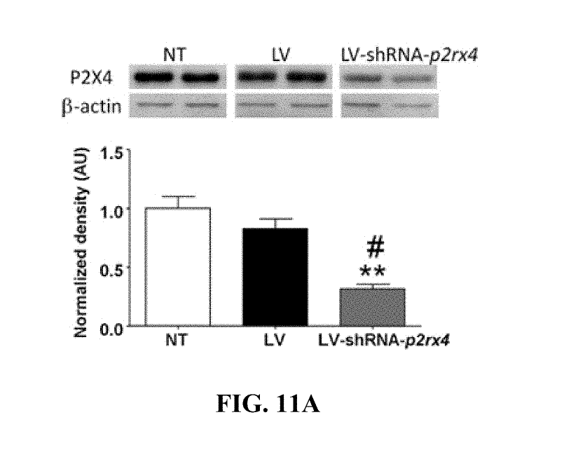

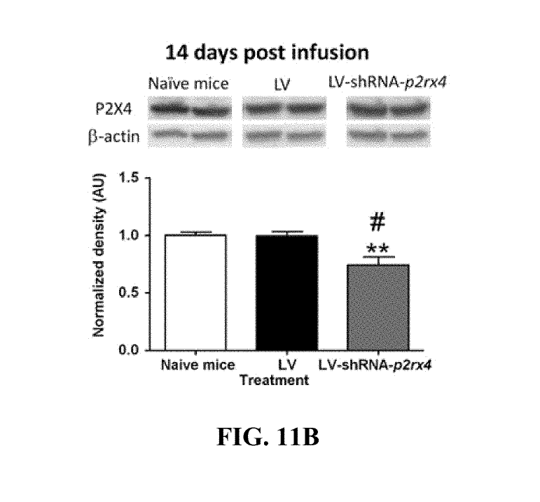

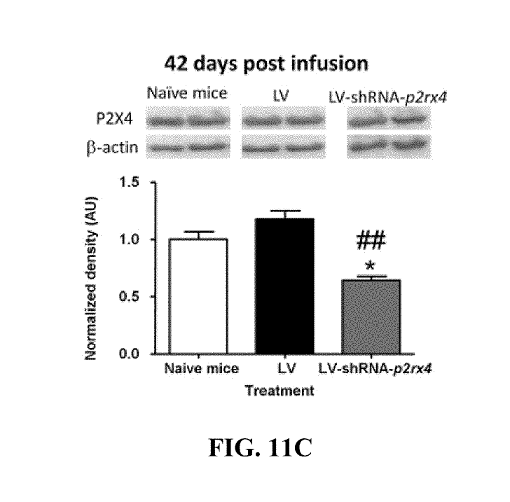

The efficiency of knockdown of P2X4Rs using a LV-shRNA strategy in BV-2 cells was investigated. As depicted in FIG. 11A, LV-shRNA treatment significantly reduced P2X4R expression (F_{(2,4)}=27.88, p<0.01) in BV-2 cells. Tukey's post hoc test confirmed that LV-shRNA treatment significantly reduced P2X4R expression as compared to untreated cells (q=9.875, p<0.01) and cells treated with LV alone (q ¼ 7.377, p<0.05).

The efficiency of LV-shRNA-p2rx4 infusion on P2X4R knockdown in the mouse striatum at 14 and 42 days post-infusion was also tested. As illustrated in FIG. 11B, LV-shRNA significantly reduced P2X4R expression in the striatum at 14 days post-infusion (F_{(2,12)}=9.266, p<0.01). Tukey's post hoc test confirmed significant reduction in P2X4R expression in mice that received LV-shRNA-p2rx4 treatment, as compared to both naïve mice (q=5.565, p<0.01) and mice that only received the LV infusion after a period of 14 days (q=4.889, p<0.05). In FIG. 11C, LV-shRNA treatment significantly reduced P2X4R expression at 42 days post-infusion (F_{(2,14)}=10.37, p<0.05), with Tukey's post hoc test confirming significant reduction in P2X4R expression in mice injected with LV-shRNA-p2rx4, in comparison to naïve controls (q=4.300, p<0.05) and mice that received LV infusion alone (q=6.435, p<0.01).

Infusion of LV alone did not have any significant effect on ethanol intake or preference in comparison to the naïve mice. There was a non-significant trend toward effect of week (F_{(4,76)}=2.35, p=0.0618) without any significant effect of treatment or week×treatment interaction for 10E intake in mice infused with LV alone relative to naïve mice. Similarly, in the context of 10E preference, there was a non-significant trend toward effect of week (F_{(4,76)}=2.03, p=0.0982) without any significant effect of treatment or week×treatment interaction. There was a significant effect of week (F_{(4,76)}=2.59, p<0.05) but not treatment, and the week×treatment interaction trended toward significance (F_{(4,76)}=2.19, p=0.0782) for water intake between the two groups. There was a non-significant trend toward effect of week (F_{(4,76)}=2.21, p=0.0753) and treatment (F_{(1,19)}=3.86, p=0.0642) on total fluid intake between the two groups of mice. However, there was a significant week×treatment interaction (F_{(4,76)}=2.75, p<0.05), with a Bonferroni post hoc test indicating reduced total fluid intake in mice receiving LV infusion relative to naïve mice at week 5 (t=3.329, p<0.01). Finally, there was a non-significant trend toward effect of week on body weight (F_{(4,76)}=2.49, p=0.0503) without any significant effect of treatment or week×treatment interaction between LV-alone infused mice and naïve mice (data not shown).

The LV-shRNA-p2rx4 Infused Mice Exhibited a Higher Ethanol Intake as Compared to Mice that Only Received LV Infusion

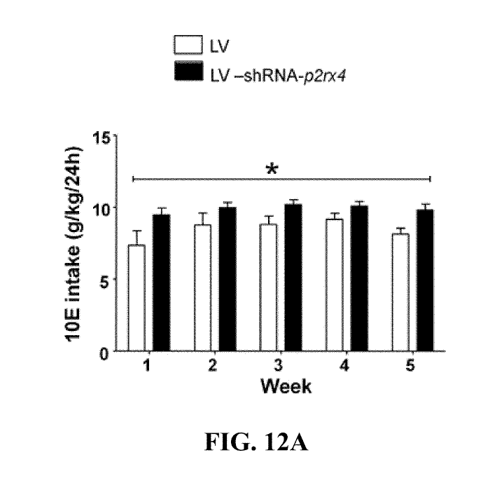

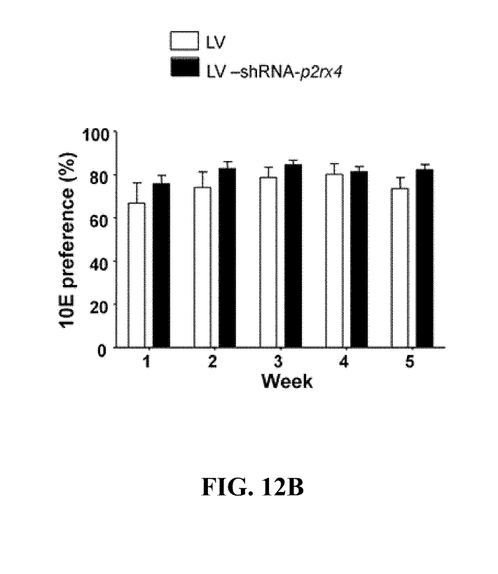

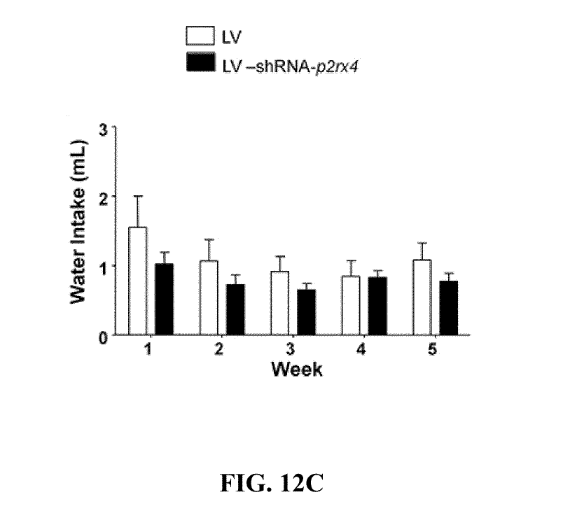

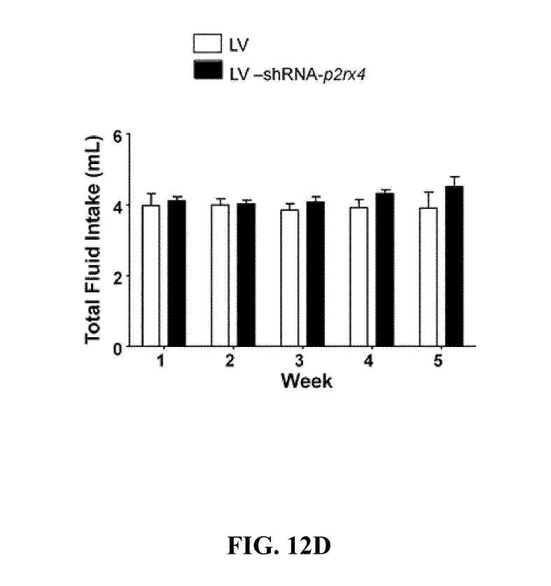

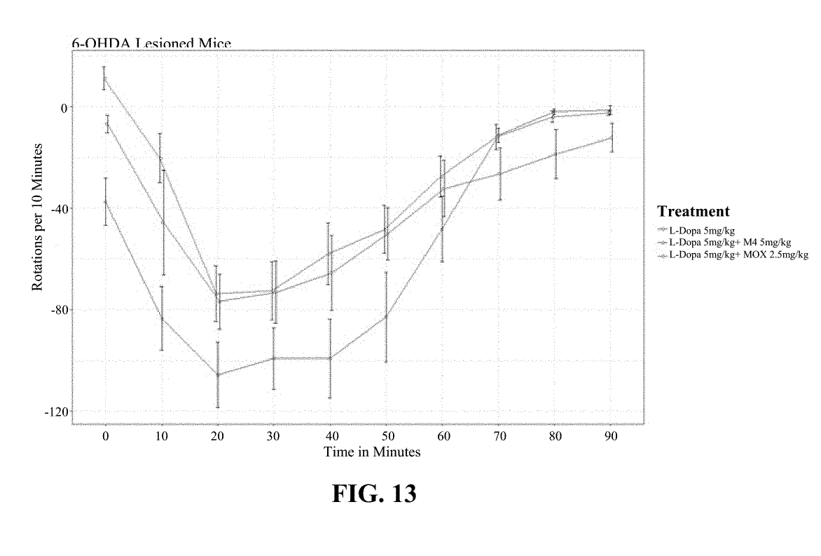

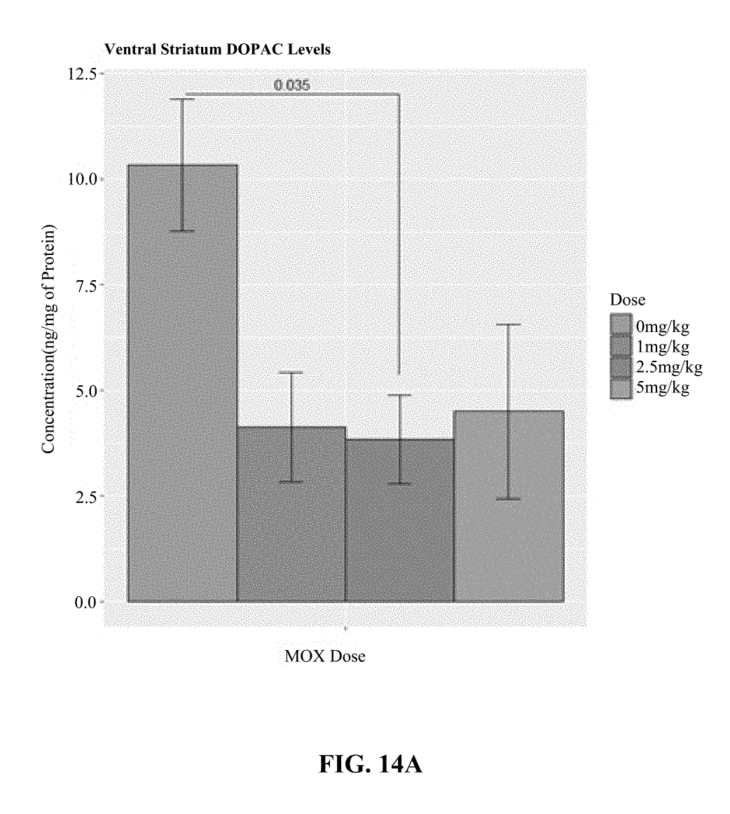

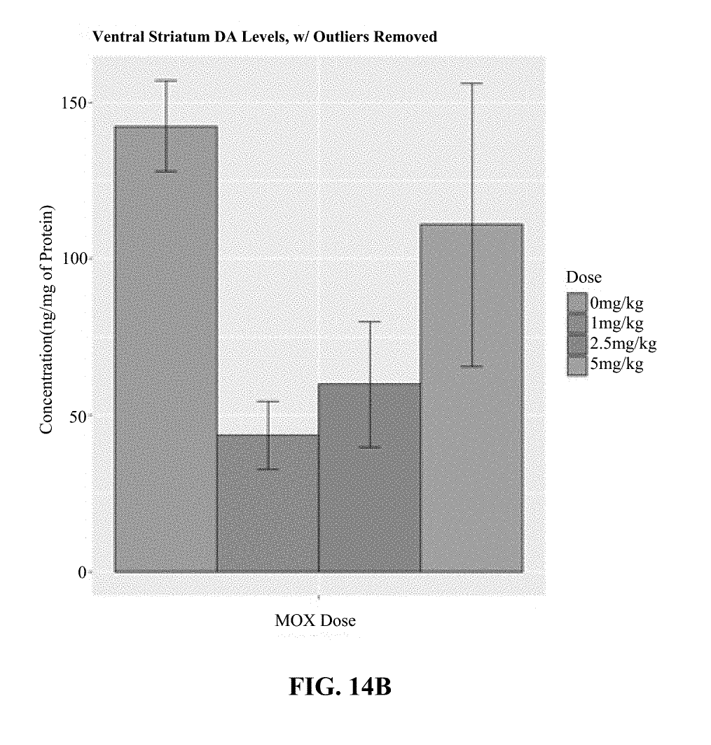

There was a significant effect of week (F_{(4,80)}=3.87, p<0.01) and treatment (F_{(1,20)}=6.08, p<0.05) on 10E intake in the LV-shRNA-p2rx4 group as compared to the group that received LV infusion, without any significant week×treatment interaction as shown in FIG. 12A. There was a significant effect of week (F_{(4,80)}=3.96, p<0.01) but not treatment or week×treatment interaction for 10E preference between the two groups as shown in FIG. 12B. Similarly, there was a significant effect of week (F_{(4,80)}=5.57, p<0.001), but not treatment or week×treatment interaction for water intake (FIG. 12C). There was no significant effect of week, treatment, or week×treatment interaction for total fluid intake (FIG. 12D). There was a significant effect of week (F_{(4,80)}=16.60, p<0.001), but not treatment on body weight between the two groups. However, there was a significant week×treatment interaction (F_{(4,80)}=3.39, p<0.05) for body weight because LV-shRNA-p2rx4 infused mice gained more weight across the 5-week period.

### Discussion

The current study investigated the role of P2X4Rs in regulation of ethanol drinking behavior. Overall, the findings support the hypothesis that P2X4Rs play an important role in the regulation of ethanol intake by demonstrating that reduced P2X4R expression results in changes in ethanol drinking behavior. Using a global knockout strategy, it was demonstrated that P2X4R KO mice exhibited significantly increased ethanol intake. The increased body weights of P2X4R KO mice may partially contribute to their increased ethanol intake, as suggested by lack of a significant change in ethanol preference. On the other hand, there was no significant change in water intake, suggesting that p2rx4 deficiency affects the mechanism of ethanol drinking, without perturbing the physiology of drinking per se. These results are in good agreement with the study discussed in Example 1, in which it was shown that male P2X4R KO mice exhibited higher ethanol intake over a period of 4 days without changes in ethanol preference or water intake as compared to their WT littermates (Wyatt, L. R. et al. (2014) Neurochemical Research 39:1127-1139).

To elucidate a direct link between P2X4R antagonism and increased ethanol consumption. A LV-shRNA methodology was employed to address this issue. The NAc core as the site for the LV-shRNA injection was targeted because this is a critical site of the dopamine (DA) mesolimbic circuitry for various drugs of abuse, including ethanol, to induce their reinforcing and rewarding effects. Moreover, P2X4Rs expression in the striatum and endogenous ATP (possibly via activation of P2XRs) may be relevant in modulation of DA neurotransmission in various regions of the mesolimbic circuitry, including the VTA and NAc. In agreement with the previous and current findings from the male P2X4R KO study, it was seen that mice with reduced P2X4R expression (via LV-shRNA-p2rx4 infusion) exhibited greater ethanol consumption relative to naïve mice and mice infused with LV alone. There were no significant changes in ethanol intake upon infusion of LV alone in relation to naïve mice, indicating that the increased ethanol intake in mice with reduced P2X4R expression is due to shRNA-mediated knockdown of P2X4Rs and not infusion of the virus alone. The coherence in findings from both the P2X4R KO and LV-shRNA-p2rx4 studies indicates that functional deletion of the p2rx4 gene increases ethanol intake.

Additionally, shRNA-mediated knockdown of P2X4Rs significantly increased ethanol preference relative to naïve mice but not mice infused with LV alone. The increased ethanol preference in LV-shRNA-p2rx4 infused mice may account for the increased ethanol intake relative to naïve mice, since there were no significant differences in body weights between these groups. Unlike in the LV-shRNA methodology, where there is knockdown of a particular gene at the adult stage, the P2X4R KO mice may exhibit neurodevelopmental adaptations to compensate for constitutive deficiency of the p2rx4 gene, and such adaptations could nullify the effect of p2rx4 knockout on ethanol preference. Moreover, the p2rx4 knockdown is in the NAc core, which is a key brain region for expression of ethanol reinforcement. Thus, potential neurodevelopmental changes in this brain region of P2X4R KO mice could significantly interfere with motivational behavior toward seeking ethanol.

The studies described herein with P2X4R KO mice coupled with the LV-shRNA in this example, support the hypothesis that P2X4Rs play an important role in regulation of ethanol intake.

### Example 3: Effect of Positive P2X4R Modulation by IVM and MOX on Dopamine (DA) Signaling

### Materials and Methods

This study examined the effects of MOX versus IVM in a unilateral model of dopamine depletion in the striatum to gain mechanistic insights into the effects of these two compounds on the dopaminergic system within the brain. The focus of this work was in the striatum of the CNS. It was believed that MOX would significantly enhance the L-DOPA induced rotations in mice with medial forebrain bundle lesions in a manner similar to IVM.

### Stereotaxic Surgery

30 male mice underwent stereotaxic surgery. 30 minutes before surgery all mice were injected with desipramine (I.P. 25 mg/kg). During surgery 2 μl of 6-Hydroxydopamine (4 mg/mL 6-OHDA) was injected into the medial forebrain bundle, at the following coordinates (relative to bregma): anterior-posterior (A/P)=−1.1; medio-lateral (M/L)=+1.3 and dorso-ventral (D/V)=−5.0. 20 mice received 6-OHDA injections and 10 mice received saline at the same coordinates. After surgery mice were allowed to recover for one week.

### Rotation

30 Minutes before testing began mice were moved into the behavior room to allow for acclimation. Mice were first given 0.1 ml saline (I.P.) and placed into a cylinder for 10 minutes to measure baseline rotations. Then mice were injected with amphetamine (I.P. 5 mg/kg) and placed into a cylinder and observed for a period of 120 minutes. During this period mice were placed in a room with only red light to prevent disruption of the animal's natural light/dark cycle. After amphetamine testing mice were returned to their cages and allowed a one week wash out period before any further behavioral test were conducted.

Rotation with MOX and IVM

Mice were given IVM (I.P. 5 mg/kg) 8 hours prior to rotational testing or mice MOX (LP. 2.5 mg/kg) 4 hours prior to rotational testing. 30 minutes before testing began mice were moved into the behavior room to allow for acclimation. Mice were then given 0.1 ml saline and placed into a cylinder for 10 minutes to establish baseline rotations. Next, mice were injected with L-Dopa (S.C. 5 mg/kg) and placed into a cylinder and observed for a period of 90 minutes. During this period mice were placed in a room with only red light to prevent disruption of the animals natural light/dark cycle. A washout period of at least 7 days was used before testing the mice with another drug using the rotational tests.

### Scoring of Rotations

All rotation tests were recorded and scored by researchers who were blinded to the treatment groups. Rotations were counted only if the mouse preformed a tight rotation, appearing as if it was chasing after its own tail. Both contralateral and ipsilateral rotations were recorded.

### Dissections and Tissue Collection

20 mice 8 week old C57/BL6 mice were obtained from Jackson Laboratory and used for tissue collection. 4 hours prior to dissection mice were given (I.P) either saline, 1 mg/kg MOX, 2.5 mg/kg MOX, or 5 mg/kg MOX. At dissection tissue was collected from the dorsal striatum, ventral striatum, prefrontal cortex, hippocampus, cortex, and cerebellum. Samples were analyzed via HPLC

### Results

MOX only slightly increased L-DOPA induced rotational behavior in mice who had unilateral medial forebrain bundle lesions. IVM significantly increased L-DOPA induced rotational behavior. Taken together, these findings suggest that IVM represents a better candidate drug when considering adjunct therapies to L-DOPA for indications such as Parkinson's disease. Biochemically, MOX significantly decreased DOPAC, a metabolite of dopamine, indicating the ability of MOX to change the dopaminergic system on its own. This suggests MOX may be able to alter dopamine metabolism. Studies are currently underway measuring the metabolite profile of IVM.

### EQUIVALENTS

It should be understood that although the present invention has been specifically disclosed by certain aspects, embodiments, refinements and optional features, modification, improvement and variation of such aspects, embodiments, and optional features can be resorted to by those skilled in the art, and that such modifications, improvements and variations are considered to be within the scope of this disclosure.

The invention has been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

### REFERENCES

1. Abduljawad K. A., Langley R. W., Bradshaw C. M. and Szabadi E. (1998). Effects of bromocriptine and haloperidol on prepulse inhibition of the acoustic startle response in man. J. Psychopharmacol. 12L-DOPA therapy, 239-245.
2. Amadio, S., Montilli, C., Picconi, B., Calabresi, P., & Volont, C. (2007). Mapping P2X and P2Y receptor proteins in striatum and substantia nigra: An immunohistological study. Purinergic Signalling, 3, 389-398.
3. Andries M., Van Damme P., Robberecht W. and Van Den Bosch L. (2007). Ivermectin inhibits AMPA receptor-mediated excitotoxicity in cultured motor neurons and extends the life span of a transgenic mouse model of amyotrophic lateral sclerosis. Neurobiol. Dis. 25, 8-16.
4. Asatryan L., Popova M., Perkins D. I., Trudell J. R., Alkana R. L. and Davies D. L. (2010). Ivermectin antagonizes ethanol inhibition in P2X4 receptors. J. Pharmacol. Exp. Ther. 334, 720-728.
5. Asatryan, L., Nam, H. W., Lee, M. R., Thakkar, M. M., Sareed Dar, M., Davies, D. L., et al. (2011). Implication of the purinergic system in alcohol use disorders. Alcoholism: Clinical and Experimental Research, 35, 584-594.
6. Asatryan, L., Popova, M., Perkins, D. I., Trudell, J. R., Alkana, R. L., & Davies, D. L. (2010). Ivermectin antagonizes ethanol inhibition in P2X4 receptors. Journal of Pharmacology and Experimental Therapeutics, 334, 720-728.
7. Asatryan, L., Popova, M., Woodward, J. J., King, B. F., Alkana, R. L., & Davies, D. L. (2008). Roles of ectodomain and transmembrane regions in ethanol and agonist action in purinergic P2X2 and P2X3 receptors. Neuropharmacology, 55, 835-843.
8. Asatryan, L., Yardley, M. M., Khoja, S., Trudell, J. R., Hyunh, N., Louie, S. G., et al. (2014). Avermectins differentially affect ethanol intake and receptor function: Implications for developing new therapeutics for alcohol use disorders. The International Journal of Neuropsychopharmacology, 17, 907-916.
9. Bailer U., Leisch F., Meszaros K. et al. (2000). Genome scan for susceptibility loci for schizophrenia. Neuropsychobiology 42, 175-182.
10. Bassareo, V., Cucca, F., Frau, R., & Di Chiara, G. (2017). Changes in dopamine transmission in the nucleus accumbens shell and core during ethanol and sucrose self-administration. Frontiers in Behavioral Neuroscience, 11, 71.
11. Baxter A. W., Choi S. J., Sim J. A. and North R. A. (2011). Role of P2X4 receptors in synaptic strengthening in mouse CA1 hippocampal neurons. Eur. J. Neurosci. 34, 213-220.
12. Behbehani, M. M. (1995). Functional characteristics of the midbrain periaqueductal gray. Progress in Neurobiology, 46, 575-605.
13. Belknap, J. K., Crabbe, J. C., & Young, E. R. (1993). Voluntary consumption of ethanol in 15 inbred mouse strains. Psychopharmacology (Berl), 112, 503-510.
14. Bertran-Gonzalez J., Bosch C., Maroteaux M., Matamales M., Herve D., Valjent E. and Girault J. A. (2008). Opposing patterns of signaling activation in dopamine D1 and D2 receptor-expressing striatal neurons in response to cocaine and haloperidol. J. Neurosci. 28, 5671-5685.
15. Blednov Y. A., Walker D., Alva H., Creech K., Findlay G. and Harris R. A. (2003). GABAA receptor alpha 1 and beta 2 subunit null mutant mice: behavioral responses to ethanol. J. Pharmacol. Exp. Ther. 305, 854-863.
16. Bortolato M., Yardley M., Khoja S., Godar S. C., Asatryan L., Finn D. A., Alkana R. L., Louie S. G. and Davies D. L. (2013). Pharmacological insights into the role of P2X4 receptors in behavioral regulation: lessons from ivermectin. Int. J. Neuropsychopharmacol. 16, 1059-1070.
17. Braff D. L. (1993). Information processing and attention dysfunctions in schizophrenia. Schizophr. Bull. 19, 233-259.
18. Brunswick D. J., Amsterdam J. D., Mozley P. D. and Newberg A. (2003). Greater availability of brain dopamine transporters in major depression shown by [99 m Tc]TRODAT-1 SPECT imaging. Am. J. Psychiatry 160, 1836-1841.
19. Buell, G., Lewis, C., Collo, G., North, R. A., & Suprenant, A. (1996). An antagonistinsensitive P2X receptor expressed in epithelia and brain. The EMBO Journal, 15, 55-62.
20. Burnstock, G. (2008). Purinergic signalling anddisorders of the central nervous system. Nature Reviews Drug Discovery, 7, 575-590. https://doi.org/10.1038/nrd2605.
21. Cador, M., Taylor, J. R., & Robbins, T. W. (1991). Potentiation of the effects of rewardrelated stimuli by dopaminergic-dependent mechanisms in the nucleus accumbens. Psychopharmacology, 104, 377-385.
22. Caseley E. A., Muench S. P., Roger S., Mao H. J., Baldwin S. A. and Jiang L. H. (2014). Non-synonymous single nucleotide polymorphisms in the P2X receptor genes: association with diseases, impact on receptor functions and potential use as diagnosis biomarkers. Int. J. Mol. Sci. 15, 13344-13371.
23. Cheung K. K., Chan W. Y. and Burnstock G. (2005). Expression of P2X purinoceptors during rat brain development and their inhibitory role on motor axon outgrowth in neural tube explant cultures. Neuroscience 133, 937-945.
24. Chizh, B. A., & Illes, P. (2001). P2X receptors and nociception. Pharmacological Reviews, 53, 553-568.
25. Corbit, L. H., Fischbach, S. C., & Janak, P. H. (2016). Nucleus accumbens core and shell are differentially involved in general and outcome-specific forms of Pavlovianinstrumental transfer with alcohol and sucrose rewards. European Journal of Neuroscience, 43, 1229-1236.
26. Davies, D. L., Kochegarov, A. A., Kuo, S. T., Kulkarni, A. A., Woodward, J. J., King, B. F., et al. (2005). Ethanol differentially affects ATP-gated P2X(3) and P2X(4) receptor subtypes expressed in Xenopus oocytes. Neuropharmacology, 49, 243-253.
27. Davies, D. L., Machu, T. K., Guo, Y., & Alkana, R. L. (2002). Ethanol sensitivity in ATPgated P2X receptors is subunit dependent. Alcoholism: Clinical and Experimental Research, 26, 773e778.
28. Dawson E., Parfitt E., Roberts Q. et al. (1995). Linkage studies of bipolar disorder in the region of the Darier's disease gene on chromosome 12q23-24.1. Am. J. Med. Genet. 60, 94-102.
29. Dawson G. R., Wafford K. A., Smith A., Marshall G. R., Bayley P. J., Schaeffer J. M., Meinke P. T. and McKernan R. M. (2000). Anticonvulsant and adverse effects of avermectin analogs in mice are mediated through the gamma-aminobutyric acid A receptor. J. Pharmacol. Exp. Ther. 295, 1051-1060.
30. Dawson, G. R., Wafford, K. A., Smith, A., Marshall, G. R., Bayley, P. J., Schaeffer, J. M., et al. (2000). Anticonvulsant and adverse effects of avermectin analogs in mice are mediated through the gamma-aminobutyric acid(A) receptor. The Journal of Pharmacology and Experimental Therapeutics, 295, 1051-1060.
31. Deumens R., Blokland A. and Prickaerts J. (2002). Modeling Parkinson's disease in rats: an evaluation of 6-OHDA lesions of the nigrostriatal pathway. Exp. Neurol. 175, 303-317.
32. Di Chiara, G. (2002). Nucleus accumbens shell and core dopamine: Differential role in behavior and addiction. Behavioural Brain Research, 137, 75-114.
33. Doherty J. M., Masten V. L., Powell S. B., Ralph R. J., Klamer D., Low M. J. and Geyer M. A. (2008). Contributions of dopamine D1, D2, and D3 receptor subtypes to the disruptive effects of cocaine on prepulse inhibition in mice. Neuropsychopharmacology 33, 2648-2656.
34. Du X., Elberger A. J., Matthews D. B. and Hamre K. M. (2012). Heterozygous deletion of NR1 subunit of the NMDA receptor alters ethanol-related behaviors and regional expression of NR2 subunits in the brain. Neurotoxicol. Teratol. 34, 177-186.
35. Duncan G. E., MoyS. S., Perez A., Eddy D. M., Zinzow W. M., Lieberman J. A., SnouwaertJ. N. and Koller B. H. (2004). Deficits in sensorimotor gating and tests of social behavior in a genetic model of reduced NMDA receptor function. Behay. Brain Res. 153, 507-519.
36. Eells J. B. (2003). The control of dopamine neuron development, function and survival: insights from transgenic mice and the relevance to human disease. Curr. Med. Chem. 10, 857-870.
37. Fauchey V., Jaber M., Caron M. G., Bloch B. and Le Moine C. (2000). Differential regulation of the dopamine D1, D2 and D3 receptor gene expression and changes in the phenotype of the striatal neurons in mice lacking the dopamine transporter. Eur. J. Neurosci. 12, 19-26.
38. Feifel D., Minassian A. and Perry W. (2009). Prepulse inhibition of startle in adults with ADHD. J. Psychiatr. Res. 43, 484-489.
39. Fox S. H. and Brotchie J. M. (2000). 5-HT(2C) receptor antagonists enhance the behavioural response to dopamine D(1) receptor agonists in the 6-hydroxydopamine-lesioned rat. Eur. J. Pharmacol. 398, 59-64.
40. Franklin B. J. and Paxinos G. (2007). The Mouse Brain in Stereotaxic Coordinates. Elsevier Academic Press, Amsterdam.
41. Franklin, K. M., Asatryan, L., Jakowec, M. W., Trudell, J. R., Bell, R. L., & Davies, D. L. (2014). P2X4 receptors (P2X4Rs) represent a novel target for the development of drugs to prevent and/or treat alcohol use disorders. Frontiers in Neuroscience, 8, 176.
42. Franklin, K. M., Hauser, S. R., Lasek, A. W., Bell, R. L., & McBride, W. J. (2015). Involvement of purinergic P2X4 receptors in alcohol intake of high-alcoholdrinking (HAD) rats. Alcoholism: Clinical and Experimental Research, 39, 2022-2031. https://doi.org/10.1111/acer.12836.
43. Franklin, K., & Paxinos, G. (2007). The mouse brain in stereotaxic coordinates. Academic Press.
44. Gendreau P. L., Petitto J. M., Petrova A., Gariepy J. and Lewis M. H. (2000). D(3) and D(2) dopamine receptor agonists differentially modulate isolation-induced social-emotional reactivity in mice. Behay. Brain Res. 114, 107-117.
45. Ghisi V., Ramsey A. J., Masri B., Gainetdinov R. R., Caron M. G. and Salahpour A. (2009). Reduced D2-mediated signaling activity and trans-synaptic upregulation of D1 and D2 dopamine receptors in mice overexpressing the dopamine transporter. Cell. Signal. 21, 87-94.
46. Girault J. A. (2012). Integrating neurotransmission in striatal medium spiny neurons. Adv. Exp. Med. Biol. 970, 407-429.
47. Gofman, L., Cenna, J. M., & Potula, R. (2014). P2X4 receptor regulates alcoholinduced responses in microglia. Journal of Neuroimmune Pharmacology, 9, 668-678.
48. Gofman, L., Fernandes, N. C., & Potula, R. (2016). Relative role of Akt, ERK and CREB in alcohol-induced microglia P2X4R receptor expression. Alcohol and Alcoholism, 51, 647-654.
49. Gonzales, R. A., Job, M. O., & Doyon, W. M. (2004). The role of mesolimbic dopamine in the development and maintenance of ethanol reinforcement. Pharmacology & Therapeutics, 103, 121-146.
50. Gu B. J., Sun C., Valova V. A., Skarratt K. K. and Wiley J. S. (2010). Identification of the promoter region of the P2RX4 gene. Mol. Biol. Rep. 37, 3369-3376.
51. Gu J. G. and MacDermott A. B. (1997). Activation of ATP P2X receptors elicits glutamate release from sensory neuron synapses. Nature 389, 749-753.
52. Gum, R. J., Wakefield, B., & Jarvis, M. F. (2012). P2X receptor antagonists for pain management: Examination of binding and physicochemical properties. Purinergic Signalling, 8(Suppl 1), 41-56.
53. Hattori M. and Gouaux E. (2012). Molecular mechanism of ATP binding and ion channel activation in P2X receptors. Nature 485, 207-212.
54. Heine C., Wegner A., Grosche J., Allgaier C., Illes P. and Franke H. (2007). P2 receptor expression in the dopaminergic system of the rat brain during development. Neuroscience 149, 165-181.
55. Hietala J., Syvalahti E., Vilkman H. et al. (1999). Depressive symptoms and presynaptic dopamine function in neuroleptic-naïve schizophrenia. Schizophr. Res. 35, 41-50.
56. Hietala J., Syvalahti E., Vuorio K. et al. (1995). Presynaptic dopamine function in striatum of neuroleptic-naive schizophrenic patients. Lancet 346, 1130-1131.
57. Hugel S. and Schlichter R. (2002). Presynaptic P2X receptors facilitate inhibitory GABAergic transmission between cultured rat spinal cord dorsal horn neurons. J. Neurosci. 20, 2121-2130.
58. Huynh, N., Arabian, N., Naito, A., Louie, S., Jakowec, M. W., Asatryan, L., & Davies, D. L. (2017, February). Preclinical development of moxidectin as a novel therapeutic for alcohol use disorder. Retrieved Aug. 15, 2017
59. Ison J. R. and Hoffman H. S. (1983). Reflex modification in the domain of startle: II. The anomalous history of a robust and ubiquitous phenomenon. Psychol. Bull. 94, 3-17.
60. Jelinkova I., Vavra V., Jindrichova M., Obsil T., Zemkova H. W., Zemkova H. and Stojilkovic S. S. (2008). Identification of P2X(4) receptor transmembrane residues contributing to channel gating and interaction with ivermectin. Pflugers Arch. 456, 939-950.
61. Jelinkova I., Yan Z., Liang Z., Moonat S., Teisinger J., Stojilkovic S. S. and Zemkova H. (2006). Identification of P2X4 receptor-specific residues contributing to the ivermectin effects on channel deactivation. Biochem. Biophys. Res. Commun. 349, 619-625.
62. Jo Y. H., Donier E., Martinez A., Garret M., Toulme E. and Boue-Grabot E. (2011). Cross-talk between P2X4 and gammaaminobutyric acid, type A receptors determines synaptic efficacy at a central synapse. J. Biol. Chem. 286, 19993-20004.
63. Jo, Y. H., & Schlichter, R. (1999). Synaptic corelease of ATP and GABA in cultured spinal neurons. Nature Neuroscience, 2, 241-245.
64. Jones I., Jacobsen N., Green E. K., Elvidge G. P., Owen M. J. and Craddock N. (2002). Evidence for familial cosegregation of major affective disorder and genetic markers flanking the gene for Darier's disease. Mol. Psychiatry 7, 424-427.
65. Khakh B. S. and North R. A. (2012). Neuromodulation by extracellular ATP and P2X receptors in the CNS. Neuron 76, 51-69.
66. Khakh B. S., Proctor W. R., Dunwiddie T. V., Labarca C. and Lester H. A. (1999). Allosteric control of gating and kinetics at P2X4 receptor channels. J. Neurosci. 19, 7289-7299.
67. Khakh, B. S. (2001). Molecular physiology of P2X receptors and ATP signalling at synapses. Nature Reviews Neuroscience, 2, 165-174.
68. Khoja, S., Shah, V., Garcia, D., Asatryan, L., Jakowec, M. W., & Davies, D. L. (2016, 08). Role of purinergic P2X4 receptors in regulating striatal dopamine homeostasis and dependent behaviors. Journal of Neurochemistry, 139(1), 134-148.
69. Kim, E. K., & Choi, E. J. (2010). Pathological roles of MAPK signaling pathways in human diseases. Biochimica et Biophysica Acta, 1802, 396-405.
70. Kimpel, M. W., Strother, W. N., McClintick, J. N., Carr, L. G., Liang, T., Edenberg, H. J., et al. (2007). Functional gene expression differences between inbred alcoholpreferring and—non-preferring rats in five brain regions. Alcohol, 41, 95-132.
71. Kinkead B., Dobner P. R., Egnatashvili V., Murray T., Deitemeyer N. and Nemeroff C. B. (2005). Neurotensin-deficient mice have deficits in prepulse inhibition: restoration by clozapine but not haloperidol, olanzapine, or quetiapine. J. Pharmacol. Exp. Ther. 315, 256-264.
72. Krause K. H., Dresel S. H., Krause J., Kung H. F. and Tatsch K. (2000). Increased striatal dopamine transporter in adult patients with attention deficit hyperactivity disorder: effects of methylphenidate as measured by single photon emission computed tomography. Neurosci. Lett. 285, 107-110.
73. Krause R. M., Buisson B., Bertrand S., Corringer P. J., Galzi J. L., Changeux J. P. and Bertrand D. (1998). Ivermectin: a positive allosteric effector of the alpha 7 meuronal nicotinic acetylcholine receptor. Mol. Pharmacol. 53, 283-294.
74. Krugel U., Kittner H., Franke H. and Illes P. (2001). Stimulation of P2 receptors in the ventral tegmental area enhances dopaminergic mechanisms in vivo. Neuropharmacology 40, 1084-1093.
75. Krügel U., Kittner H., Franke H. and Illes P. (2003). Purinergic modulation of neuronal activity in the mesolimbic dopaminergic system in vivo. Synapse 47, 134-142.
76. Krügel, U., Kittner, H., & Illes, P. (2001). Mechanisms of adenosine 50-triphosphateinduced dopamine release in the rat nucleus accumbens in vivo. Synapse, 39, 222-232.
77. Kumari V., Mulligan O. F., Cotter P. A., Poon L., Toone B. K., Checkley S. A. and Gray J. A. (1998). Effects of single oral administrations of haloperidol and d-amphetamine on prepulse inhibition of the acoustic startle reflex in healthy male volunteers. Behay. Pharmacol. 9, 567-576.
78. Le T. K., Villeneuve P., Ramjaun A. R., McPherson P. S., Beaudet A. and Séguéla P. (1998). Sensory presynaptic and widespread somatodendritic immunolocalization of central ionotropic P2XATP receptors. Neuroscience 83, 177-190.
79. Li, C., Aguayo, L., Peoples, R. W., & Weight, F. F. (1993). Ethanol inhibits a neuronal ATP-gated ion channel. Molecular Pharmacology, 44, 871e875. Li, C., Peoples, R. W., & Weight, F. F. (1994). Alcohol action on a neuronal membrane receptor: Evidence for a direct interaction with the receptor protein. Proceedings of the National Academy of Sciences of the United States of America, 91, 8200-8204.
80. Li, C., Peoples, R. W., & Weight, F. F. (1998). Ethanol-induced inhibition of a neuronal P2X purinoceptor by an allosteric mechanism. British Journal of Pharmacology, 123, 1-3.
81. Lipina T. V., Niwa M., Jaaro-Peled H., Fletcher P. J., Seeman P., Sawa A. and Roder J. C. (2010). Enhanced dopamine function in DISC1-L100P mutant mice: implications for schizophrenia. Genes Brain Behay. 9, 777-789.
82. Litten, R. Z., Egli, M., Heilig, M., Cui, C., Fertig, J. B., Ryan, M. L., et al. (2012). Medications development to treat alcohol dependence: A vision for the next decade. Addiction Biology, 17, 513-527.
83. Lorca, R. A., Rozas, C., Loyola, S., Moreira-Ramos, S., Zeise, M. L., Kirkwood, A., et al. (2011). Zinc enhances long-term potentiation through P2X receptor modulation in the hippocampal CA1 region. The European Journal of Neuroscience, 33, 1175-1185.
84. McBride, W. J., Kimpel, M. W., McClintick, J. N., Ding, Z. M., Hyytia, P., Colombo, G., et al. (2012). Gene expression in the ventral tegmental area of 5 pairs of rat lines selectively bred for high or low ethanol consumption. Pharmacology, Biochemistry, and Behavior, 102, 275-285.
85. McClintick, J. N., McBride, W. J., Bell, R. L., Ding, Z. M., Liu, Y., Xuei, X., et al. (2016). Gene expression changes in glutamate and GABA-A receptors, neuropeptides, ion channels, and cholesterol synthesis in the periaqueductal gray following binge-like alcohol drinking by adolescent alcohol-preferring (P) rats. Alcoholism: Clinical and Experimental Research, 40, 955-968.
86. McCool, B. A. (2011). Ethanol modulation of synaptic plasticity. Neuropharmacology, 61, 1097-1108.
87. Ménez, C., Sutra, J., Prichard, R., & Lespine, A. (2012, 11). Relative Neurotoxicity of Ivermectin and Moxidectin in Mdrlab (−/−) Mice and Effects on Mammalian GABA(A) Channel Activity. PLoS Neglected Tropical Diseases, 6(11). doi:10.1371/journal.pntd.0001883
88. Middaugh, L. D., & Kelley, B. M. (1999). Operant ethanol reward in C57BL/6 mice: Influence of gender and procedural variables. Alcohol, 17, 185-194.
89. Moxidectin for system suitability Y0000747. (n.d.). Retrieved Aug. 17, 2017
90. North R. A. (2002). Molecular physiology of P2X receptors. Physiol. Rev. 82, 1013-1067.
91. Ortiz R., Ulrich H., Zarate C. A., Jr and Machado-Vieira R. (2015). Purinergic system dysfunction in mood disorders: a key target for developing improved therapeutics. Prog. Neuropsychopharmacol. Biol. Psychiatry 57, 117-131.
92. Ostrovskaya, O., Asatryan, L., Wyatt, L., Popova, M., Li, K., Peoples, R. W., et al. (2011). Ethanol is a fast channel inhibitor of P2X4 receptors. The Journal of Pharmacology and Experimental Therapeutics, 337, 171-179.
93. Pankratov, Y., Lalo, U., Krishtal, O. A., & Verkhratsky, A. (2009). P2X receptors and synaptic plasticity. Neuroscience, 158, 137-148.
94. Pearlson G. D., Wong D. F., Tune L. E. et al. (1995). In vivo D2 dopamine receptor density in psychotic and nonpsychotic patients with bipolar disorder. Arch. Gen. Psychiatry 52, 471-477.
95. Perez V., Catafau A. M., Corripio I., Martin J. C. and Alvarez E. (2003). Preliminary evidence of striatal D2 receptor density as a possible biological marker of prognosis in naive schizophrenic patients. Prog. Neuropsychopharmacol. Biol. Psychiatry 27, 767-770.
96. Perry W., Minassian A., Feifel D. and Braff D. L. (2001). Sensorimotor gating deficits in bipolar disorder patients with acute psychotic mania. Biol. Psychiatry 50, 418-424.
97. Popova M., Trudell J., Li K., Alkana R., Davies D. and Asatryan L. (2013). Tryptophan 46 is a site for ethanol and ivermectin action in P2X4 receptors. Purinergic Signal. 9, 621-632.
98. Popova, M., Asatryan, L., Ostrovskaya, O., Wyatt, L. R., Li, K., Alkana, R. L., et al. (2010). A point mutation in the ectodomain-transmembrane 2 interface eliminates the inhibitory effects of ethanol in P2X4 receptors. Journal of Neurochemistry, 112, 307-317.
99. Potucek, Y. D., Crain, J. M., & Watters, J. J. (2006). Purinergic receptors modulate MAP kinases and transcription factors that control microglial inflammatory gene expression. Neurochemistry International, 49, 204-214.
100. Priel A. and Silberberg S. D. (2004). Mechanism of ivermectin facilitation of human P2X4 receptor channels. J. Gen. Physiol. 123, 281-293.
101. Ralph R. J., Paulus M. P., Fumagalli F., Caron M. G. and Geyer M. A. (2001). Prepulse inhibition deficits and perseverative motor patterns in dopamine transporter knock-out mice: differential effects of D1 and D2 receptor antagonists. J. Neurosci. 21, 305-313.
102. Ralph-Williams R. J., Lehmann-Masten V. and Geyer M. A. (2003). Dopamine D1 rather than D2 receptor agonists disrupt prepulse inhibition of startle in mice. Neuropsychopharmacology 28, 108-118.
103. Ralph-Williams R. J., Lehmann-Masten V., Otero-Corchon V., Low M. J. and Geyer M. A. (2002). Differential effects of direct and indirect dopamine agonists on prepulse inhibition: a study in D1 and D2 receptor knock-out mice. J. Neurosci. 22, 9604-9611.
104. Rodriguiz R. M., Chu R., Caron M. G. and Wetsel W. C. (2004). Aberrant responses in social interaction of dopamine transporter knockout mice. Behay. Brain Res. 148, 185-198.
105. Schwarting R. K. and Huston J. P. (1996). The unilateral 6-hydroxydopamine lesion model in behavioral brain research. Analysis of functional deficits, recovery and treatments. Prog. Neurobiol. 50, 275-331.
106. Seeman P. (2013a). Are dopamine D2 receptors out of control in psychosis? Prog. Neuropsychopharmacol. Biol. Psychiatry 46, 146-152.
107. Seeman P. (2013b). Schizophrenia and dopamine receptors. Eur. Neuropsychopharmacol. 23, 999-1009.
108. Seeman P., Schwarz J., Chen J. F. et al. (2006). Psychosis pathways converge via D2high dopamine receptors. Synapse 60, 319-346.
109. Shan Q., Haddrill J. L. and Lynch J. W. (2001). Ivermectin, an unconventional agonist of the glycine receptor chloride channel. J. Biol. Chem. 276, 12556-12564.
110. Sim J. A., Chaumont S., Jo J., Ulmann L., Young M. T., Cho K., Buell G., North R. A. and Rassendren F. (2006). Altered hippocampal synaptic potentiation in P2X4 knock-out mice. J. Neurosci. 26, 9006-9009.
111. Soto, F., Garcia-Guzman, M., Gomez-Hernandez, J. M., Hollmann, M., Karschin, C., & Stiihmer, W. (1996). P2X4: An ATP-activated ionotropic receptor cloned from rat brain. Proceedings of the National Academy of Sciences of the United States of America, 93, 3684-3688.
112. Svenningsson P., Nishi A., Fisone G., Girault J. A., Nairn A. C. and Greengard P. (2004). DARPP-32: an integrator of neurotransmission. Annu. Rev. Pharmacol. Toxicol. 44, 269-296.
113. Swerdlow N. R., Keith V. A., Braff D. L. and Geyer M. A. (1991). Effects of spiperone, raclopride, SCH 23390 and clozapine on apomorphine inhibition of sensorimotor gating of the startle response in the rat. J. Pharmacol. Exp. Ther. 256, 530-536.
114. Swerdlow N. R., Weber M., Qu Y., Light G. A. and Braff D. L. (2008). Realistic expectations of prepulse inhibition in translational models for schizophrenia research. Psychopharmacology 199, 331-388.
115. Tabakoff, B., Saba, L., Printz, M., Flodman, P., Hodgkinson, C., Goldman, D., et al. (2009). Genetical genomic determinants of alcohol consumption in rats and humans. BMC Biology, 7, 70.
116. Tsuda, M., Koizumi, S., Kita, A., Shigemoto, Y., Ueno, S., & Inoue, K. (2000). Mechanical allodynia caused by intraplantar injection of P2X receptor agonist in rats: Involvement of heteromeric P2X2/3 receptor signaling in capsaicin-insensitive primary afferent neurons. The Journal of Neuroscience, 20, RC90.
117. Tsuda, M., Masuda, T., Tozaki-Saitoh, H., & Inoue, K. (2013). P2X4 receptors and neuropathic pain. Frontiers in Cellular Neuroscience, 7, 191.
118. Ulmann, L., Hatcher, J. P., Hughes, J. P., Chaumont, S., Green, P. J., Conquet, F., et al. (2008). Up-regulation of P2X4 receptors in spinal microglia after peripheral nerve injury mediates BDNF release and neuropathic pain. The Journal of Neuroscience, 28, 11263-11268.
119. Ungerstedt U. (1971). Postsynaptic supersensitivity after 6-hydroxydopamine induced degeneration of the nigro-striatal dopamine system. Acta Physiol. Scand. Suppl. 367, 69-93.
120. Volter C., Riedel M., Wostmann N. et al. (2012). Sensorimotor gating and D2 receptor signalling: evidence from a molecular genetic approach. Int. J. Neuropsychopharmacol. 15, 1427-1440.
121. Weight, F. F., Li, C., & Peoples, R. W. (1999). Alcohol action on membrane ion channels gated by extracellular ATP (P2X receptors). Neurochemistry International, 35, 143-152.
122. Wolinsky T. D., Swanson C. J., Smith K. E., Zhong H., Borowsky B., Seeman P., Branchek T. and Gerald C. P. (2007). The Trace Amine 1 receptor knockout mouse: an animal model with relevance to schizophrenia. Genes Brain Behay. 6, 628-639.
123. Wyatt, L. R., Finn, D. A., Khoja, S., Yardley, M. M., Asatryan, L., Alkana, R. L., et al. (2014). Contribution of P2X4 receptors to ethanol intake in male C57BL/6 mice. Neurochemical Research, 39(6), 1127-1139.
124. Wyatt, L. R., Godar, S. C., Khoja, S., Jakowec, M. W., Alkana, R. L., Bortolato, M., et al. (2013). Sociocommunicative and sensorimotor impairments in male P2X4-deficient mice. Neuropsychopharmacology, 38, 1993-2002.
125. Xiao D., Cassin J. J., Healy B., Burdett T. C., Chen J. F., Fredholm B. B. and Schwarzschild M. A. (2011). Deletion of adenosine A(1) or A ((2)A) receptors reduces L-3,4 dihydroxyphenylalanine-induced dyskinesia in a model of Parkinson's disease. Brain Res. 1367, 310-318.
126. Xiao, C., Zhou, C., Li, K., Davies, D. L., & Ye, J. H. (2008). Purinergic type 2 receptors at GABAergic synapses on ventral tegmental area dopamine neurons are targets for ethanol action. The Journal of Pharmacology and Experimental Therapeutics, 327, 196-205.
127. Xiong, K. M., Li, C., & Weight, F. F. (2001). Differential modulation by short chain and long chain n-alcohols of rat P2X 4 receptors expressed in Xenopus oocytes. Alcoholism: Clinical and Experimental Research, 25, 7A.
128. Xiong, K., Li, C., & Weight, F. F. (2000). Inhibition by ethanol of rat P2X(4) receptors expressed in Xenopus oocytes. British Journal of Pharmacology, 130, 1394-1398.
129. Xu, J., Bernstein, A. M., Wong, A., Lu, X. H., Khoja, S., Yang, X. W., et al. (2016). P2X4 receptor reporter mice: Sparse brain expression and feeding-related presynaptic facilitation in the arcuate nucleus. The Journal of Neuroscience, 36, 8902-8920.
130. Yardley, M. M., Wyatt, L., Khoja, S., Asatryan, L., Ramaker, M. J., Finn, D. A., et al. (2012). Ivermectin reduces alcohol intake and preference in mice. Neuropharmacology, 63(2), 190-201.
131. Yoneyama, N., Crabbe, J. C., Ford, M. M., Murillo, A., & Finn, D. A. (2008). Voluntary ethanol consumption in 22 inbred mouse strains. Alcohol, 42, 149-160.
132. Zemkova, H., Kucka, M., Li, S., Gonzalez-Iglesias, A. E., Tomic, M., & Stojilkovic, S. S. (2010). Characterization of purinergic P2X4 receptor channels expressed in anterior pituitary cells. American Journal of Physiology. Endocrinology and Metabolism, 298, E644-E651.
133. Zhan L., Kerr J. R., Lafuente M. J., Maclean A., Chibalina M. V., Liu B., Burke B., Bevan S. and Nasir J. (2011). Altered expression and coregulation of dopamine signalling genes in schizophrenia and bipolar disorder. Neuropathol. Appl. Neurobiol. 37, 206-219.
134. Zhou F. C., Zhang J. K., Lumeng L. and Li T. K. (1995). Mesolimbic dopamine system in alcohol-preferring rats. Alcohol 12, 403-412.

What is claimed is:

1. A method for the treatment of one or more of: Parkinson's disease (PD); PD symptoms; movement disorders; and neurodegenerative diseases linked to changes in dopamine, dopamine signaling, or dopamine expression, in a subject in need thereof comprising administering to the subject an effective amount of an agent that potentiates dopaminergic neurotransmission.

2. The method of claim 1, wherein the PD symptoms are selected from tremors, rigidity and bradykinesia.

3. The method of claim 1, wherein the neurodegenerative diseases linked to changes in dopamine are selected from the group of: encephalitis, concussions, dopamine-responsive dystonia, and Parkinsonian syndrome.

4. The method of claim 1, wherein the agent that potentiates dopaminergic neurotransmission is selected from the group of: ivermectin (IVM), an IVM analogue, a P2X4R allosteric modulator, and an avermectin compound.

5. The method of claim 4, wherein the IVM analogue is a compound of the formula: 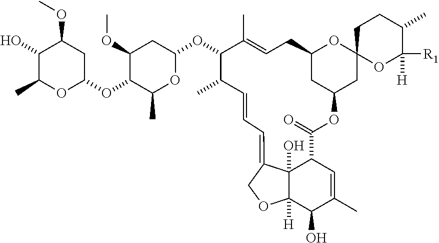
wherein R₁ is a C₁-C₁₀ alkyl, or a pharmaceutically acceptable salt or solvate thereof.

6. The method of claim 5, wherein R₁ is methyl, ethyl, isopropyl, n-propyl, isobutyl, sec-butyl, or n-butyl.

7. The method of claim 5, wherein R₁ is isopropyl, n-propyl, isobutyl, or sec-butyl.

8. The method of claim 1, wherein the agent that potentiates dopaminergic neurotransmission comprises or consists essentially of IVM and/or moxidectin (MOX), or a pharmaceutically acceptable salt or solvate of each thereof.

9. The method of claim 8, further comprising administering to the subject an effective amount of an L-dopamine therapy.

10. The method of claim 9, wherein the effective amount of the L-dopamine therapy is administered prior to, subsequent to, or concurrently with, the agent that potentiates dopaminergic neurotransmission.

11. The method of claim 10, wherein the L-dopamine therapy is L-DOPA.

12. The method of claim 11, further comprising administering to the subject an effective amount of carbidopa or benserazide.

13. The method of claim 12, wherein L-DOPA and carbidopa are administered in a mass ratio of 4 to 1 or 10 to 1.

14. The method of claim 1, wherein the subject is a mammal.

15. The method of claim 14, wherein the mammal is a human.

16. A method for selecting a candidate agent for the treatment of one or more of: Parkinson's disease (PD); PD symptoms; and neurodegenerative diseases linked to changes in dopamine, dopamine signaling, or dopamine expression, in a subject in need thereof, the method comprising contacting a compound with a cell expressing a P2X4 receptor under conditions for binding of the candidate agent to the P2X4 receptor and selecting the compound as the candidate agent if it potentiates P2X4 receptor activity.

17. The method of claim 16, wherein the cell is a neural cell.

18. The method of claim 16, wherein the cell is a cell modified to express a P2X4 receptor.

19. The method of claim 1, wherein the agent that potentiates dopaminergic neurotransmission comprises or consists essentially of IVM, or a pharmaceutically acceptable salt or solvate of each thereof.

20. The method of claim 1, wherein the agent that potentiates dopaminergic neurotransmission comprises or consists essentially of MOX, or a pharmaceutically acceptable salt or solvate of each thereof.
